(19) United States
(12) Patent Application Publication
(10) Pub. No.: US 2025/0204869 A1
(43) Pub. Date: Jun. 26, 2025

(54) ALERT OPTIMIZATION IN MULTI-RECEIVER ENVIRONMENTS

(71) Applicant: Dexcom, Inc., San Diego, CA (US)

(72) Inventors: Brian C. SMITH, San Diego, CA (US); Peter B. HEDLUND, San Diego, CA (US); Jason R. CALABRESE, San Diego, CA (US)

(21) Appl. No.: 18/984,806

(22) Filed: Dec. 17, 2024

Related U.S. Application Data
(60) Provisional application No. 63/612,923, filed on Dec. 20, 2023.

(51) Int. Cl. A61B 5/00; A61B 5/145

(52) U.S. Cl. CPC A61B 5/746; A61B 5/14532; A61B 5/14546; A61B 2560/045; A61B 2560/0462

(57) ABSTRACT

In one embodiment, a method of alert optimization includes receiving, at a first device, one or more analyte measurements produced by an analyte sensor worn by a patient. The method also includes determining, at the first device, a first unique identifier for a first alert based on the one or more analyte measurements. The method also includes comparing, at the first device, the first unique identifier for the first alert to a stored record that includes unique identifiers for historical alerts. The method also includes conditionally suppressing the first alert on the first device based on the comparing.

### CROSS-REFERENCE TO RELATED APPLICATIONS

This application claims priority to and benefit of U.S. Provisional Patent Application No. 63/612,923, filed Dec. 20, 2023, which is hereby expressly incorporated by reference herein in its entirety as if fully set forth below and for all applicable purposes.

### INTRODUCTION

Many diseases or conditions are dependent upon maintaining analyte levels within an acceptable range. As one example, diabetes is a metabolic condition affecting hundreds of millions of people. For these people, monitoring blood glucose levels and regulating those levels to be within an acceptable range is important not only to mitigate long-term issues such as heart disease and vision loss, but also to avoid the effects of hyperglycemia and hypoglycemia. Maintaining blood glucose levels within an acceptable range can be challenging, as glucose levels are almost constantly changing over time and in response to everyday events, such as eating or exercising.

Advances in medical technologies have enabled development of various systems for monitoring analytes such as blood glucose, including continuous analyte monitoring (CAM) systems such as continuous glucose monitoring (CGM) systems, which measure and record glucose concentrations in substantially real-time. CAM systems are important tools for users of these systems to ensure that measured analyte values are within the acceptable range.

### SUMMARY

In some embodiments, one general aspect includes a method of alert optimization. The method includes receiving, at a first device, one or more analyte measurements produced by an analyte sensor worn by a patient. The method also includes determining, at the first device, a first unique identifier for a first alert based on the one or more analyte measurements. The method also includes comparing, at the first device, the first unique identifier for the first alert to a stored record that includes unique identifiers for historical alerts. The method also includes conditionally suppressing the first alert on the first device based on the comparing.

In some embodiments, another general aspect includes a system for alert optimization. The system includes an analyte sensor configured to generate measurements associated with an analyte level of a patient. The system also includes a device in communication with the analyte sensor. The device is configured to receive one or more analyte measurements produced by the analyte sensor. The device is further configured to determine a unique identifier for a first alert based on the one or more analyte measurements. The device is further configured to compare the unique identifier for the first alert to a stored record comprising unique identifiers for historical alerts. The device is further configured to conditionally suppress the first alert on the device based on the comparing.

In some embodiments, another general aspect includes a computer-program product. The computer-program product includes a non-transitory computer-usable medium having computer-readable program code embodied therein. The computer-readable program code is adapted to be executed to implement a method of alert optimization. The method includes receiving one or more analyte measurements produced by an analyte sensor worn by a patient. The method also includes determining a first unique identifier for a first alert based on the one or more analyte measurements. The method also includes comparing the first unique identifier for the first alert to a stored record may include unique identifiers for historical alerts. The method also includes conditionally suppressing the first alert on the device based on the comparing.

### BRIEF DESCRIPTION OF THE DRAWINGS

FIG. 1A illustrates an example of a therapy management system, in accordance with certain embodiments.

FIG. 1B illustrates an example analyte sensor system including an example continuous analyte sensor(s) with sensor electronics, in accordance with certain embodiments.

FIG. 2 illustrates example inputs and example outputs that are generated based on the inputs, in accordance with certain embodiments.

FIG. 3 illustrates an example of a system for sharing a patient's health data between devices and/or with one or more other users, in accordance with certain embodiments.

FIG. 4 illustrates an example of a process for executing alert deduplication at a device, in accordance with certain embodiments.

FIG. 5A illustrates an example of a process for executing alert deduplication in response to an alert determination at a device, in accordance with certain embodiments.

FIG. 5B illustrates an example of a process for preventing redundant processing of analyte data, in accordance with certain embodiments.

FIG. 6 illustrates an example of a process for executing alert deduplication in response to receiving an alert notification from another device, in accordance with certain embodiments.

FIG. 7 illustrates an example of a process for synchronizing alert state between devices, in accordance with certain embodiments.

FIG. 8 illustrates an example of a process for adjusting alert timing in response to an updated alert state, in accordance with certain embodiments.

FIG. 9 illustrates an example of a process for adapting to device modes that impact alert presentation, in accordance with certain embodiments.

FIG. 10A illustrates an example of a process for delaying transmission of an updated alert state, in accordance with certain embodiments.

FIG. 10B illustrates an example of a process for transmitting cached alert states upon reconnection with a device, in accordance with certain embodiments.

FIG. 11 illustrates an example of a process for synchronizing settings between devices, in accordance with certain embodiments.

FIG. 12 illustrates an example of a process for generating and implementing alert analytics, in accordance with certain embodiments.

FIG. 13 illustrates an example of a process for adapting settings to a current user, in accordance with certain embodiments.

FIG. 14 is a block diagram depicting a computer system configured for optimizing transmission of analyte data in multi-receiver environments, in accordance with certain embodiments.

### DETAILED DESCRIPTION

In a continuous analyte monitoring system, an analyte sensor measures analyte levels of a patient and communicates the raw sensor measurements to a transmitter, which can then transmit corresponding analyte values to the host receiver device (e.g., a smartphone) for presentation, alerting, and/or storage. The patient can thereby use their device to monitor their health substantially in real-time.

In certain aspects, multiple receiver devices (e.g., display devices such as a smartphone, smartwatch, tablet, augmented reality (AR) and/or virtual reality (VR) headset, etc.) can each be configured to directly communicate with, and obtain analyte measurements from, a single analyte sensor. The obtained analyte measurements can then be used by each receiver device to generate and present alerts. These receiver devices may also communicate information (including analyte and alert information) to each other for review and presentation by the device receiving such information. This may result in redundant/duplicate alerts that are problematic or confusing to users. Alerts can be duplicates and not be necessary on all displays.

In some aspects, the above-mentioned alerting redundancy can be due to individual alert generation at the receiver devices. For example, the receiver devices can each independently determine whether to generate and present an alert to a user based on the same or similar criteria, such as whether the analyte measurements satisfy certain low or high thresholds. Accordingly, the same or similar alerts may be independently generated and presented on each of the devices.

In some aspects, the above-mentioned alerting redundancy can be due to sharing of alerts between devices. For example, the receiver devices can be configured to share analyte data and/or alerts with each other. For example, a smartphone can generate and present an alert that a high or low threshold has been crossed and, additionally, can communicate the alert to a smartwatch connected thereto. Thereafter, the smartwatch can present the same alert on that device. However, the smartwatch may also independently generate and present the same alert that is received from the smartphone. This can result in the smartwatch displaying the same alert multiple times to a user. In one example, even after the user has acknowledged an initially presented first instance of an alert, the user may then be presented with a second instance of the same alert at a later time. This may result in alert fatigue and general confusion on the part of the user.

Although it can be advantageous to have multiple ways to capture a user's attention, it may be that multiple receiver devices present multiple alerts for the same event or condition, where each of these redundant alerts may need to be viewed by the user as if it represents distinct information. For example, for a single instance of a high or low threshold being crossed, a smartphone and smartwatch may each independently generate and present similar alerts. Additionally, one or both of the smartphone and smartwatch may share the individually generated alerts with each other, which may result in one or more further alerts being presented to the user. According to this example, it may be that four alerts for the same event or condition are presented to the user.

Due to the aforementioned redundancies, users may be confused by, and/or lose trust in, the presented alerts and/or develop alert fatigue. As a result, users may be hesitant to act on the alerts. This hesitance may negatively impact the health of the patient providing the analyte data. In some cases, the user's confusion may result in the patient being given the wrong treatment, such as a wrong amount treatment (e.g., an excess amount of glucose or insulin), based on a mistaken belief that the redundancy of alerts across multiple devices indicates extreme urgency.

Further to the above, implementations that support multiple devices for alerting may maintain different settings, such as different settings related to appearance and/or functionality. In an example, the settings can include alert settings. The alert settings may provide for alerting a user, for example, if the user's analyte values exceed a threshold value for a threshold period of time. The alert settings may also provide for whether and how an alert is presented, such as sound, volume, vibration, and silence, where each may vary for different times and/or analyte values. In certain implementations, some or all of the foregoing settings may be configurable by a user. However, the user may have to enter preferred settings multiple times for multiple devices (e.g., for each of a smartwatch and smartphone), which can interfere with the usage of these devices for monitoring health.

In response to the above problems, in certain aspects described herein, receiver devices connected to an analyte sensor (e.g., smartphone and smartwatch) can each independently execute alert de-duplication prior to presenting an alert at the device. For each alert, an alert identifier can be deterministically generated, such that the same identifier is generated for all alerts that result from the same trigger, regardless of where (e.g., at which device) the alert is generated. For example, the alert identifier may be a combination of an alert type (e.g., high, low, etc.) and a time associated with an analyte measurement that resulted in the alert. The time associated with the analyte measurement may be, for example, a timestamp indicative of date and time, a sensor tick time (e.g., a time value that increments from when the analyte sensor is first instantiated), or the like. In addition, or alternatively, the alert identifier can include information to distinguish the alert by sensor data type (e.g., for implementations involving multiple sensors such as glucose, lactate, ketone and/or the like). For example, the alert identifier can include an identifier of a sensor type, a hash of analyte data resulting in the alert, and/or the like. In various aspects, each device may share alerts generated thereon with other devices (e.g., smartphone to smartwatch, smartwatch to smartphone, smartphone to insulin pump, etc.), for example, by sending alert notifications that include the alert identifiers.

In certain aspects, the receiver devices can each maintain, or have access to, a stored record of historical alerts previously processed at the device, for example, due to generation at that device or to receipt of an alert notification from another device. The stored record can be updated with a corresponding alert identifier and/or other data each time an alert is processed at the device. In certain aspects, as part of the alerting process, each device can cause an alert to be presented or suppressed based on whether an alert with the same alert identifier has already been presented on that device (e.g., based on a comparison with the stored record of historical alerts previously processed at the device). In this way, each receiver device can independently execute alert deduplication in response to an alert generated at the device and/or in response to an alert notification received from another receiver device.

In further response to the above problems, in certain aspects, receiver devices can share updates to alert states with other receiver devices. The updates can include or indicate, for example, a user response to a given alert, such as whether the alert has been acknowledged by the user (e.g., via a button press or swipe), whether the alert has been responded to with treatment, and/or the like. For example, one device (referred to as Device #1 for clarity), such as a smartwatch, can determine an updated alert state (e.g., that an alert has been acknowledged) and can share the updated alert state with another device (referred to as Device #2 for clarity), such as a smartphone. The updated alert state can include, for example, an alert identifier, as discussed above, and a timestamp (e.g., a time of acknowledgement). In response, Device #2 can suppress the alert at that device, clear the alert at that device, adjust timing for re-presentation of the alert at that device, and/or the like.

In some aspects, the sharing of alert states can be adapted or optimized in response to device states or modes that impact alert presentation. In an example, continuing the above illustration with Device #1 and Device #2, if Device #2 is in a do-not-disturb (DND) mode, or a similar mode that prevents alert presentation, and configurable conditions are present (e.g., the situation is urgent and/or alerts are not being acknowledged at Device #1 and/or Device #2), Device #1 can indicate the elevated urgency to Device #2, thereby causing Device #2 to escalate an alarm to overcome the DND mode and present the alert at that device. In another example, if communication between Device #1 and Device #2 is interrupted, Device #1 can cache an updated alert state for transmission, and then transmit the updated alert state to Device #2, for example, upon reestablishment of a connection therewith.

In further response to the above problems, in certain aspects, receiver devices can synchronize settings by maintaining settings states in relation to time stamps. When settings, such as alert settings, are changed at one device, that device can save an updated settings state in association with a timestamp of the change. The updated settings state and associated time stamp can be shared with other devices, thereby synchronizing settings between multiple devices. In various cases, each device can determine and implement a most recent settings state based on the timestamp and/or roll back to an earlier settings state based on similar timestamps.

In further response to the above problems, in certain aspects, alert analytics can be generated, for example, based on stored alert records on devices (e.g., the stored record of historical alerts referenced above). In some aspects, the analytics may be used to perform, for example, personalized optimization of settings to decrease alert fatigue and increase user engagement with alerts. In some aspects, the analytics may be used to dynamically establish or change a priority device for alert presentation based on user behavior (e.g., based on time and/or location).

In further response to the above problems, in certain aspects, receiver devices can modify alerting functionality based on a current user of the device. For example, if a smartphone determines that it is currently being used by a non-target user, such as a child of the patient, the smartphone can temporarily modify alerting functionality, for example, by overriding alert suppression features, implementing additional alert sharing, etc. For example, acknowledgements and/or other alert state updates at the smartphone may be ignored, not recorded, and/or not substantively acted upon. In this way, when the smartphone is returned to the target user, previous alerts may still be viewable and/or re-presentation of alerts may still occur. In addition, or alternatively, alert state updates may not be shared with other devices. In addition, or alternatively, any suppression of sharing of alerts with other devices may be overridden (e.g., turned off).

Advantageously, in certain aspects, various solutions described herein can reduce the number of redundant alerts presented to users across devices (e.g., due to the utilization of deterministic alert identifiers), thereby conserving resources and improving user engagement. Further, in certain aspects, various solutions described herein enable more accurate synchronization of alerts and alert states between devices (e.g., due to the utilization of deterministic alert identifiers), thereby improving the monitoring of responses to alerts and the timing of future alerts. Additionally, in certain aspects, various solutions described herein can improve the reliability and consistency of real-time health alerts by intelligently synchronizing alerts and settings (e.g., settings related to appearance and/or functionality). In addition, in certain aspects, the improved user engagement can increase the likelihood that the user (e.g., patient) will respond to presented alerts, which will facilitates timely rectification of critical conditions indicated by the alerts and generally improves a physical health of the patient.

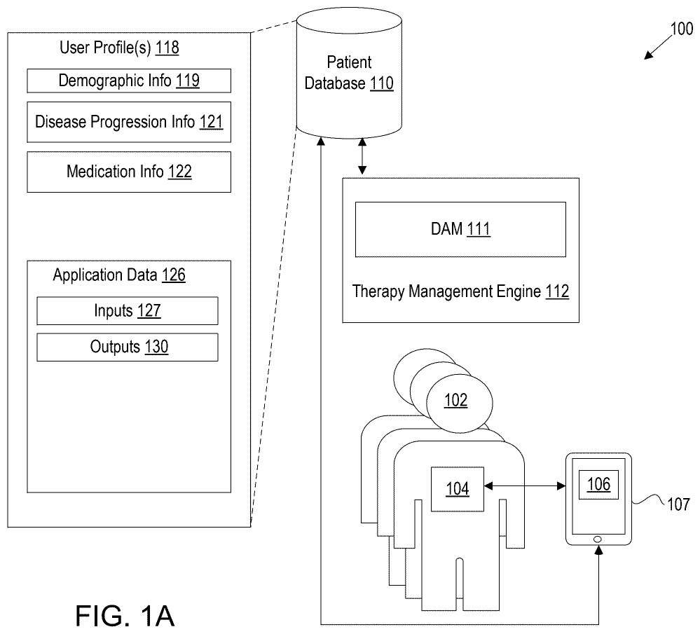

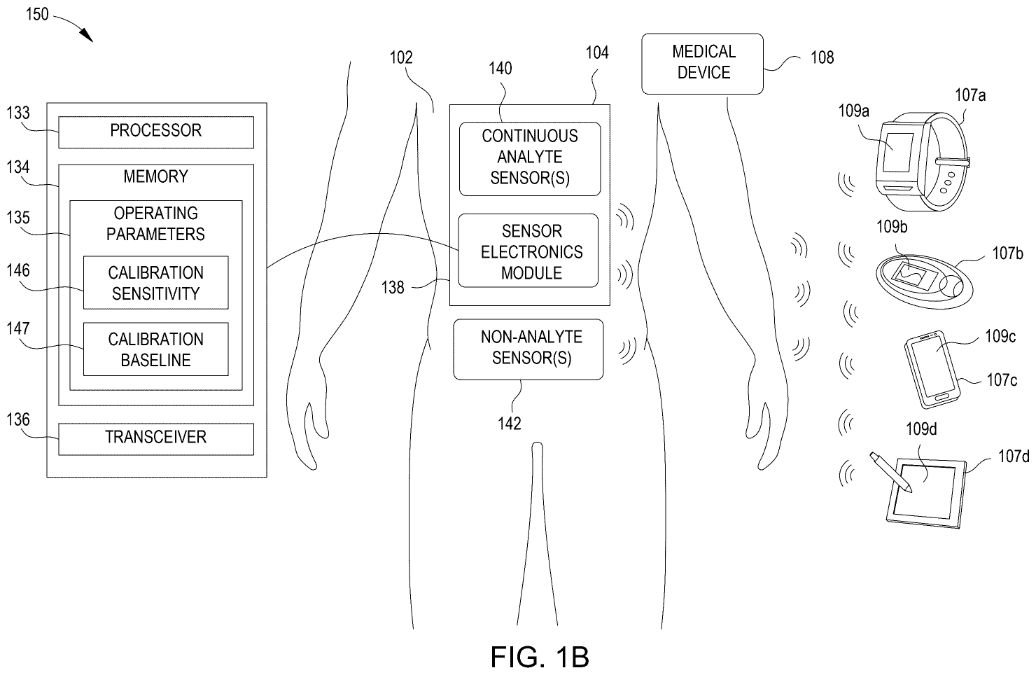

The techniques described herein for alert optimization are described more fully herein with respect to FIGS. 1A-B, 2-4, 5A-B, 6-9, 10A-B, and 12-13 below. Note that although certain aspects herein are periodically described with respect to the management of diabetes, a continuous glucose monitoring (CGM) system, a CGM application, and the transmission of analyte measurements and/alerts related thereto, the protocols and techniques described herein are similarly applicable to any type of health management system that includes any type of analyte sensor (e.g., lactate sensor, ketone sensor, etc.), any type of health management application, and/or transmission of any type of analyte data. For example, a lactate sensor may be used to monitor for events indicative of sepsis based on lactate thresholds, or a ketone sensor may be used to monitor for ketoacidosis based on ketone thresholds. Additionally, note that, hereinafter, although certain aspects described herein may periodically refer to an analyte monitoring device performing various techniques described herein for transmitting analyte data, it is the transmitter in the analyte monitoring device that performs the various techniques described herein for transmitting analyte data.

As used herein, the term “continuous” analyte monitoring refers to monitoring one or more analytes in a fully continuous, semi-continuous, periodic manner, which results in a data stream of analyte values over time. A data stream of analyte values over time is what allows for meaningful data and insights to be derived using the algorithms described herein for alert optimization. In other words, single point-in-time measurements collected as a result of a patient visiting their health care professional every few months results in sporadic data points (e.g., that are, at best, months apart in timing) that cannot form the basis of any meaningful data or insight to be derived. As such, without the continuous analyte monitoring system of the embodiments herein, it is simply impossible to continuously perform alert optimization, as described herein.

Further, the data stream of analyte values collected over time, with the continuous analyte monitoring system presented herein, include real-time analyte values, which allows for deriving meaningful data and insight in real-time using the systems and algorithms described herein. The derived real-time data and insight in turn allows for alert optimization. Real-time analyte values herein refer to analyte values that become available and actionable within seconds or minutes of being produced as a result of at least one sensor electronics module of the continuous analyte monitoring system (1) converting sensor current(s) (i.e., analog electrical signals) generated by the continuous analyte sensor(s) into sensor count values, (2) calibrating the count values to generate at least glucose and/or other analyte concentration values using calibration techniques described herein to account for the sensitivity of the continuous analyte sensor(s), and (3) transmitting measured glucose and/or other analyte concentration data, including glucose and/or other analyte concentration values, to a display device via wireless connection.

For example, the at least one sensor electronics module may be configured to sample the analog electrical signals at a particular sampling period (or rate), such as every 1 second (1 Hz), 5 seconds, 10 seconds, 30 seconds, 1 minute, 3 minutes, 5 minutes, etc., and to transmit the measured glucose and/or other analyte concentration data to a display device at a particular transmission period (or rate), which may be the same as (or longer than) the sampling period, such as every 1 minute (0.016 Hz), 5 minutes, 10 minutes, etc.

The real-time analyte data that is continuously generated by the continuous analyte monitoring system described herein, therefore, allows the therapy management system herein to perform alert optimization, in real-time, which is technically impossible to perform using existing or conventional techniques or systems. Further, because of the real-time nature of this data, it is also humanly impossible to continuously process a real-time data stream of analyte values over time to derive meaningful data and insight using the algorithms and systems described herein to perform alert optimization. In other words, deriving meaningful data and insight from a stream of real-time data that is continuously generated, processed, calibrated, and analyzed, using the algorithms and systems described herein, is not a task that can be mentally performed. For example, executing the algorithms described in relation to FIGS. 1A-B, 2-4, 5A-B, 6-9, 10A-B, and 12-13, in real-time and on a continuous basis, which would involve using a stream of real-time data that is continuously generated by a patient's continuous analyte monitoring system and/or significantly large amount of population data (e.g., hundreds or thousands of data points for each one of thousands or millions of patients in the patient population) is not a task that can be mentally performed, especially in real-time at times.

Further, certain embodiments herein are directed to a technical solution to a technical problem associated with analyte sensor systems. In particular, each analyte sensor system that is manufactured by a sensor manufacturer might perform slightly differently. As such, there might be inconsistencies between sensors and the measurements they generate once in use. Accordingly, certain embodiments herein are directed to determining the performance of an analyte sensor system during a manufacturing calibration process (in vitro), which includes quantifying certain sensor operating parameters, such as a calibration slope (also known as calibration sensitivity), a calibration baseline, etc.

Generally, calibration sensitivity refers to the amount of electrical current produced by an analyte sensor of an analyte sensor system when immersed in a predetermined amount of a measured analyte. The amount of electrical current may be expressed in units of picoAmps (pA) or counts. The amount of measured analyte may be expressed as a concentration level in units of milligrams per deciliter (mg/dL), and the calibration sensitivity may be expressed in units of pA/(mg/dL) or counts/(mg/dL). The calibration baseline refers to the amount of electrical current produced by the analyte sensor when no analyte is detected, and may be expressed in units of pA or counts.

The calibration sensitivity, calibration baseline, and other information related to the sensitivity profile for the analyte sensor system may be programmed into the sensor electronics module of the analyte sensor system during the manufacturing process, and then used to convert the analyte sensor electrical signals into measured analyte concentration levels. For example, the calibration slope (calibration sensitivity) may be used to predict an initial in vivo sensitivity (M₀) and a final in vivo sensitivity (M_{f}), which are programmed into the sensor electronics module and used to convert the analyte sensor electrical signals into measured analyte concentration levels.

In certain embodiments, during in vivo use, the sensor electronics module of an analyte sensor system samples the analog electrical signals produced by the analyte sensor to generate analyte sensor count values, and then determines the measured analyte concentration levels based on the analyte sensor count values, the initial in vivo sensitivity (M₀), and the final in vivo sensitivity (M_{f}). For example, measured analyte concentration levels may be determined using a sensitivity function M(t) that is based on the initial in vivo sensitivity (M₀) and the final in vivo sensitivity (M_{f}). The sensitivity function M(t) may expressed in several different ways, such as a simple correction factor that is not dependent on elapsed time (tᵢ) of in vivo use, a linear relationship between sensitivity and time (tᵢ), an exponential relationship between sensitivity and time (tᵢ), etc. Equation 1 presents one technique for determining a measured analyte concentration level (ACL) from an analyte sensor count value (count) at a time tᵢ:

$$ACL = count / M ( {t}_{i} ) \qquad Eq . 1$$

A calibration baseline (baseline) may also be used to determine a measured analyte concentration level (ACL) from an analyte sensor count value (count) at a time tᵢ, and Equation 2 presents one technique:

$$ACL = ( count - baseline ) / M ( {t}_{i} ) \qquad Eq . 2$$

FIG. 1A illustrates an example of a therapy management system 100 for alert optimization, in accordance with certain embodiments of the disclosure. The therapy management system 100 may be utilized for generating and presenting information related to user health, for example, using various user interfaces associated with system 100. Each user of system 100, such as user 102, may interact with a mobile health application, such as mobile health application (“application”) 106 (e.g., a diabetes intervention application that provides therapy management guidance), and/or a health monitoring device, such as an analyte sensor system 104 (e.g., a glucose monitoring system). User 102, in certain embodiments, may be the patient or, in some cases, the patient's caregiver. In the embodiments described herein, the user is assumed to be the patient for simplicity only, but is not so limited. As shown, system 100 may include an analyte sensor system 104, a display device 107 that executes application 106, a therapy management engine 112, and a user database 110.

Analyte sensor system 104 may be configured to generate time-series data, such as analyte measurements (e.g., sensor data), for the user 102, e.g., on a continuous basis, and transmit the analyte measurements to the display device 107 for use by application 106. In some embodiments, the analyte sensor system 104 may transmit the analyte measurements to the display device 107 through a wireless connection (e.g., Bluetooth connection). In certain embodiments, display device 107 is a smart phone. However, in certain embodiments, display device 107 may instead be any other type of computing device such as a laptop computer, a smartwatch, a tablet, or any other computing device capable of executing application 106.

Note that, while in certain examples the analyte sensor system 104 is assumed to be a glucose monitoring system, analyte sensor system 104 may operate to monitor one or more additional or alternative analytes. As discussed, the term “analyte” as used herein is a broad term, and is to be given its ordinary and customary meaning to a person of ordinary skill in the art (and is not to be limited to a special or customized meaning), and refers without limitation to a substance or chemical constituent in the body or a biological sample (e.g., bodily fluids, including, blood, serum, plasma, interstitial fluid, cerebral spinal fluid, lymph fluid, ocular fluid, saliva, oral fluid, urine, excretions, or exudates).

Analytes can include naturally occurring substances, artificial substances, metabolites, and/or reaction products. In some embodiments, the analyte measured and used by the devices and methods described herein may include albumin, alkaline phosphatase, alanine transaminase, aspartate aminotransferase, bilirubin, blood urea nitrogen, calcium, CO2, chloride, creatinine, glucose, gamma-glutamyl transpeptidase, hematocrit, lactate, lactate dehydrogenase, magnesium, oxygen, pH, phosphorus, potassium, ketones, sodium, total protein, uric acid, metabolic markers, and/or drugs.

Other analytes are contemplated as well, including but not limited to acetaminophen, dopamine, ephedrine, terbutaline, ascorbate, uric acid, oxygen, d-amino acid oxidase, plasma amine oxidase, xanthine oxidase, NADPH oxidase, alcohol oxidase, alcohol dehydrogenase, pyruvate dehydrogenase, diols, Ros, NO, bilirubin, cholesterol, triglycerides, gentisic acid, ibuprophen, L-Dopa, methyl dopa, salicylates, tetracycline, tolazamide, tolbutamide, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments.

The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, Advanced Glycation End Products (AGEs) and 5-hydroxyindoleacetic acid (FHIAA).

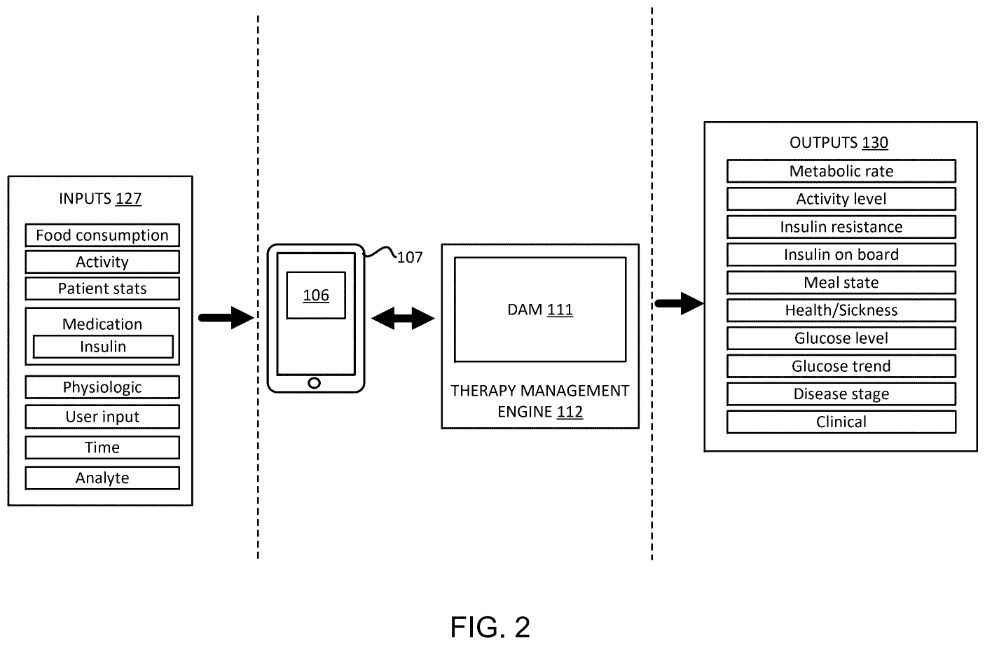

Application 106 may be a mobile health application that is configured to receive and analyze time-series data, including analyte measurements, from the analyte sensor system 104 and/or other devices, as described in greater detail relative to FIGS. 1B and 2. In some embodiments, application 106 may transmit analyte measurements received from the analyte sensor system 104 to a user database 110 (and/or the therapy management engine 112), and the user database 110 (and/or the therapy management engine 112) may store the analyte measurements in a user profile 118 of user 102 for processing and analysis, for example, by the therapy management engine 112. In some embodiments, application 106 may store the analyte measurements in a user profile 118 of user 102 locally for processing and analysis, for example, by the therapy management engine 112.

In certain embodiments, therapy management engine 112 refers to a set of software instructions with one or more software modules, including a data analysis module (DAM) 111. In some embodiments, therapy management engine 112 executes entirely on one or more computing devices in a private or a public cloud. In some other embodiments, therapy management engine 112 executes partially on one or more local devices, such as display device 107 (e.g., via application 106) and/or analyte sensor system 104, and partially on one or more computing devices in a private or a public cloud. In some other embodiments, therapy management engine 112 executes entirely on one or more local devices, such as display device 107 (e.g., via application 106) and/or analyte sensor system 104.

In certain embodiments, DAM 111 of therapy management engine 112 may be configured to receive and/or process a set of inputs 127 (described in more detail below) (also referred to herein as “input data”) to determine one or more outputs 130 (also referred to herein as “metrics data”). Inputs 127 may be stored in the user profile 118 in the user database 110. DAM 111 can fetch inputs 127 from the user database 110 and compute a plurality of outputs 130 which can then be stored as application data 126 in the user profile 118. Such outputs 130 may include health-related metrics.

In certain embodiments, application 106 is configured to take as input information relating to user 102 and store the information in a user profile 118 for user 102 in user database 110. For example, application 106 may obtain and record user 102's demographic info 119, disease progression info 121, and/or medication info 122 in user profile 118. In certain embodiments, demographic info 119 may include one or more of the user's age, body mass index (BMI), ethnicity, gender, etc. In certain embodiments, disease progression info 121 may include information about the user 102's disease, such as, for diabetes, whether the user is Type I, Type II, pre-diabetes, or whether the user has gestational diabetes. In certain embodiments, disease progression info 121 also includes the length of time since diagnosis, the level of disease control, level of compliance with disease management therapy, predicted pancreatic function, other types of diagnosis (e.g., heart disease, obesity) or measures of health (e.g., heart rate, exercise, stress, sleep, etc.), and/or the like. In certain embodiments, medication info 122 may include information about the amount and type of medication taken by user 102, such as insulin or non-insulin diabetes medications and/or non-diabetes medication taken by user 102.

In certain embodiments, application 106 may obtain demographic info 119, disease progression info 121, and/or medication info 122 from the user 102 in the form of user input or from other sources. In certain embodiments, as some of this information changes, application 106 may receive updates from the user 102 or from other sources. In certain embodiments, user profile 118 associated with the user 102, as well as other user profiles associated with other users are stored in a user database 110, which is accessible to application 106, as well as to the therapy management engine 112, over one or more networks (not shown).

In certain embodiments, application 106 collects inputs 127 through user 102 input and/or a plurality of other sources, including analyte sensor system 104, other applications running on display device 107, and/or one or more other sensors and devices. In certain embodiments, such sensors and devices include one or more of, but are not limited to, an insulin pump, other types of analyte sensors, sensors or devices provided by display device 107 (e.g., accelerometer, camera, global positioning system (GPS), heart rate monitor, etc.) or other user accessories (e.g., a smartwatch), or any other sensors or devices that provide relevant information about the user 102. In certain embodiments, user profile 118 also stores application configuration information indicating the current configuration of application 106, including its features and settings.

User database 110, in some embodiments, refers to a storage server that may operate in a public or private cloud. User database 110 may be implemented as any type of data store, such as relational databases, non-relational databases, key-value data stores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed.

User database 110 may include other user profiles 118 associated with a plurality of other users served by therapy management system 100. More particularly, similar to the operations performed with respect to the user 102, the operations performed with respect to these other users may utilize an analyte monitoring system, such as analyte sensor system 104, and also interact with the same application 106, copies of which execute on the respective display devices of the other users 102. For such users, user profiles 118 are similarly created and stored in user database 110.

FIG. 1B is a diagram 150 conceptually illustrating an example continuous analyte sensor system 104 including example continuous analyte sensor(s) with sensor electronics, in accordance with certain aspects of the present disclosure. For example, system 104 may be configured to continuously monitor one or more analytes of a patient, in accordance with certain aspects of the present disclosure.

Analyte sensor system 104 in the illustrated embodiment includes sensor electronics module 138 and one or more continuous analyte sensor(s) 140 (individually referred to herein as continuous analyte sensor 140 and collectively referred to herein as continuous analyte sensors 140) associated with sensor electronics module 138. Sensor electronics module 138 may be in wireless communication (e.g., directly or indirectly) with one or more of display devices 107a, 107b, 107c, and 107d. In certain embodiments, sensor electronics module 138 may also be in wireless communication (e.g., directly or indirectly) with one or more medical devices, such as medical devices 108 (individually referred to herein as medical device 108 and collectively referred to herein as medical devices 108), and/or one or more other non-analyte sensors 142 (individually referred to herein as non-analyte sensor 142 and collectively referred to herein as non-analyte sensor 142).

In certain embodiments, a continuous analyte sensor 140 may comprise one or more sensors for detecting and/or measuring analyte(s). The continuous analyte sensor 140 may be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. In certain embodiments, the continuous analyte sensor 140 may be configured to continuously measure analyte levels of a patient using one or more techniques, such as enzymatic techniques, chemical techniques, physical techniques, electrochemical techniques, spectrophotometric techniques, polarimetric techniques, calorimetric techniques, iontophoretic techniques, radiometric techniques, immunochemical techniques, and the like. The term “continuous,” as used herein, can mean fully continuous, semi-continuous, periodic, etc. In certain aspects, the continuous analyte sensor 140 provides a data stream indicative of the concentration of one or more analytes in the patient. The data stream may include raw data signals, which are then converted into a calibrated and/or filtered data stream used to provide estimated analyte value(s) to the patient.

In certain embodiments, the continuous analyte sensor 140 may be a multi-analyte sensor, configured to continuously measure multiple analytes in a patient's body. For example, in certain embodiments, the continuous multi-analyte sensor 140 may be a single sensor configured to measure lactate, glucose, ketones (e.g., 3-beta-hydroxybutyrate, acetoacetate, acetone, etc.), glycerol, and/or free fatty acids in the patient's body.

In certain embodiments, one or more multi-analyte sensors may be used in combination with one or more single analyte sensors. As an illustrative example, a multi-analyte sensor may be configured to continuously measure lactate and glucose and may, in some cases, be used in combination with an analyte sensor configured to measure only ketones or only potassium. Information from each of the multi-analyte sensor(s) and single analyte sensor(s) may be combined to provide therapy management support using methods described herein. In further embodiments, other non-contact and or periodic or semi-continuous, but temporally limited, measurements for physiological information may be integrated into the system such as by including weight scale information or non-contact heart rate monitoring from a sensor pad under the patient while in a chair or bed, through an infra-red camera detecting temperature and/or blood flow patterns of the patient, and/or through a visual camera with machine vision for height, weight, or other parameter estimation without physical contact.

In certain embodiments, the continuous analyte sensor(s) 140 may comprise a percutaneous wire that has a proximal portion coupled to the sensor electronics module 138 and a distal portion with several electrodes, such as a measurement electrode and a reference electrode. The measurement (or working) electrode may be coated, covered, treated, embedded, etc., with one or more chemical molecules that react with a particular analyte, and the reference electrode may provide a reference electrical voltage. The measurement electrode may generate the analog electrical signal, which is conveyed along a conductor that extends from the measurement electrode to the proximal portion of the percutaneous wire that is coupled to the sensor electronics module 138. After the continuous analyte monitoring system 104 has been applied to epidermis of the patient, continuous analyte sensor(s) 140 penetrates the epidermis, and the distal portion extends into the dermis and/or subcutaneous tissue under epidermis. Other configurations of continuous analyte sensor(s) 140 may also be used, such as a multi-analyte sensor that includes multiple measurement electrodes, each generating an analog electrical signal that represents the concentration levels of a particular analyte.

Generally, a single-analyte sensor generates an analog electrical signal that is proportional to the concentration level of a particular analyte. Similarly, each multi-analyte sensor generates multiple analog electrical signals, and each analog electrical signal is proportional to the concentration level of a particular analyte. As an illustrative example, continuous analyte sensor 140 may include a single-analyte sensor configured to measure lactate concentration levels, and another single-analyte sensor configured to measure glucose concentration levels of the patient. As another illustrative example, continuous analyte sensor(s) 140 may include a single-analyte sensor configured to measure glucose concentration levels, and one or more multi-analyte sensors configured to measure lactate concentration levels, ketone concentration levels, creatinine concentration levels, etc. As yet another illustrative example, continuous analyte sensor(s) 140 may include a multi-analyte sensor configured to measure lactate concentration levels, glucose concentration levels, ketone concentration levels, creatinine concentration levels, etc. Accordingly, continuous analyte sensor(s) 140 is configured to generate at least one analog electrical signal that is proportional to the concentration level of a particular analyte, and sensor electronics module 138 is configured to convert the analog electrical signal into an analyte sensor count values, calibrate the analyte sensor count values based on the sensitivity profile of the continuous analyte sensor(s) 140 to generate measured analyte concentration levels, and transmit the measured analyte concentration level data, including the measured analyte concentration levels, to a display device, such as display devices 107b, 107c, and/or 107d, via a wireless connection. For example, sensor electronics module 138 may be configured to sample the analog electrical signal at a particular sampling period (or rate), such as every 1 second (1 Hz), 5 seconds, 10 seconds, 30 seconds, 1 minute, 3 minutes, 5 minutes, etc., and to transmit the measured analyte concentration data to the display device at a particular transmission period (or rate), which may be the same as (or longer than) the sampling period, such as every 1 minute (0.016 Hz), 5 minutes, 10 minutes, 30 minutes, at the conclusion of the wear period, etc. Depending on the sampling and transmission periods, the measured analyte concentration data transmitted to the display device include at least one measured analyte concentration level having an associated time tag, sequence number, etc.

In certain embodiments, continuous analyte sensor(s) 140 may incorporate a thermocouple within, or alongside, the percutaneous wire to provide an analog temperature signal to the sensor electronics module 138, which may be used to correct the analog electrical signal or the measured analyte data for temperature. In other embodiments, the thermocouple may be incorporated into the sensor electronics module 138 above the adhesive pad, or, alternatively, the thermocouple may contact the epidermis of the patient through openings in the adhesive pad.

In certain embodiments, the sensor electronics module 138 includes, inter alia, processor 133, storage element or memory 134, wireless transmitter/receiver (transceiver) 136, one or more antennas coupled to wireless transceiver 136, analog electrical signal processing circuitry, analog to-digital (A/D) signal processing circuitry, digital signal processing circuitry, a power source for continuous analyte sensor(s) 140 (such as a potentiostat), etc.

Processor 133 may be a general-purpose or application-specific microprocessor, an application-specific integrated circuit (ASIC), a field programmable gate array (FPGA), etc., that executes instructions to perform control, computation, input/output, etc, functions for the sensor electronics module 138. Processor 133 may include a single integrated circuit, such as a micro processing device, or multiple integrated circuit devices and/or circuit boards working in cooperation to accomplish the appropriate functionality. In certain embodiments, processor 133, memory 134, wireless transceiver 136, the A/D signal processing circuitry, and the digital signal processing circuitry may be combined into a system-on-chip (SoC).

Generally, processor 133 may be configured to sample the analog electrical signal using the A/D signal processing circuitry at regular intervals (such as the sampling instant or period) to generate analyte sensor count values based on the analog electrical signals produced by the continuous analyte sensor(s) 140, calibrate the analyte sensor count values based on the sensitivity profile of the continuous analyte sensor(s) 140 to generate measured analyte concentration levels, and generate measured analyte data from the measured analyte concentration levels, generate sensor data packages that include, inter alia, the measured analyte concentration level data. Processor 133 may store the measured analyte concentration level data in memory 134, and generate the sensor data packages at regular intervals (such as the transmission period) for transmission by wireless transceiver 136 to a display device, such as display devices 107b, 107c, 107d, and/or 107a. Processor 133 may also add additional data to the sensor data packages, such as supplemental sensor information that includes a sensor identifier, a sensor status, temperatures that correspond to the measured analyte data, etc. The sensor data packages are then wirelessly transmitted over a wireless connection to the display device. In certain embodiments, the wireless connection is a Bluetooth or BLE connection. In such embodiments, the sensor data packages are transmitted in the form of Bluetooth or BLE data packets to the display device.

In certain aspects, the transceiver 136 maintains, in the memory 134, a connection list that indicates each device currently connected thereto. In certain aspects, the connection list can also be maintained in other storage locations (e.g., in a cloud computing environment, on each individual display device, etc.). A device may be identified as “currently connected” to the transceiver 136 if the device has successfully engaged in the wireless or wired communication of data (e.g., by requesting and/or receiving at least a predetermined amount of analyte data) with the transceiver 136 via one or more predetermined communication protocols within a predetermined period of time from the current time, has performed and successfully completed one or more wired or wireless transmission security protocols with the transceiver 136 within a predetermined period of time from the current time, etc. In certain aspects, the transceiver 136 can advertise on a regular interval (e.g., every five minutes), such that devices are removed from the connection list after a predetermined number of connections are missed (e.g., one, two, three, etc.). The connection list may include, for each device indicated therein, an identifier of the device (e.g., device identifier, user assigned identifier, transceiver 136 assigned identifier, etc.), a timestamp of last connection, signal strength, an indication of any recent transmission errors, and/or other information.

In various embodiments, memory 134 may include volatile and nonvolatile medium. For example, memory 134 may include combinations of random access memory (RAM), dynamic RAM (DRAM), static RAM (SRAM), read only memory (ROM), flash memory, cache memory, and/or any other type of non-transitory computer-readable medium. Memory 134 may store one or more analyte sensor system applications, modules, instruction sets, etc. for execution by processor 133, such as instructions to generate measured analyte data from the analyte sensor count values, etc.

Memory 134 may also store certain sensor operating parameters 135, such as a calibration slope (or calibration sensitivity), a calibration baseline, etc. In particular, the calibration sensitivity, calibration baseline, and other information related to the sensitivity profile for the sensor electronics module 138 may be programmed into the sensor electronics module 138 during the manufacturing process, and then used to convert the analyte sensor electrical signals into measured analyte concentration levels. For example, as discussed above, the calibration slope may be used to predict an initial in vivo sensitivity (M₀) and a final in vivo sensitivity (M_{f}), which are stored in memory 134 and used to convert the analyte sensor electrical signals into measured analyte concentration levels. In certain embodiments, calibration sensitivity (M_{CC}) 146 and/or calibration baseline 147 may be stored in memory 134.

In certain embodiments, sensor electronics module 138 includes electronic circuitry associated with measuring and processing the continuous analyte sensor data, including prospective algorithms associated with processing and calibration of the sensor data. Sensor electronics module 138 can be physically connected to continuous analyte sensor(s) 140 and can be integral with (non-releasably attached to) or releasably attachable to continuous analyte sensor(s) 140. Sensor electronics module 138 may include hardware, firmware, and/or software that enable measurement of levels of analyte(s) via continuous analyte sensor(s) 140. For example, sensor electronics module 138 can include a potentiostat, a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 107b, 107c, 107d, and/or 107a are configured for displaying displayable sensor data, including analyte data, which may be transmitted by sensor electronics module 138. Each of display devices 107b, 107c, 107d, or 107a may include a display such as a touchscreen display 109b, 109c, 109d, and/or 109a for displaying sensor data to a patient or other user and/or for receiving inputs from the patient. For example, a graphical user interface (GUI) may be presented to the patient for such purposes. In certain embodiments, the display devices may include other types of user interfaces such as a voice user interface instead of, or in addition to, a touchscreen display for communicating sensor data to the patient of the display device and/or for receiving patient inputs. Display devices 107a, 107b, 107c, and 107d may be examples of display device 107 illustrated in FIG. 1 used to display sensor data to a patient of the system of FIG. 1 and/or to receive input from the patient.

In certain embodiments, one, some, or all of the display devices are configured to display or otherwise communicate (e.g., verbalize) the sensor data as it is communicated from the sensor electronics module (e.g., in a customized data package that is transmitted to display devices based on their respective preferences), without any additional prospective processing required for calibration and real-time display of the sensor data.

The plurality of display devices may include a custom display device specially designed for displaying certain types of displayable sensor data associated with analyte data received from sensor electronics module. In certain embodiments, the plurality of display devices may be configured for providing alerts/alarms based on the displayable sensor data. Display device 107b is an example of such a custom device. In certain embodiments, one of the plurality of display devices is a smartphone, such as display device 107c which represents a mobile phone, using a commercially available operating system (OS), and configured to display a graphical representation of the continuous sensor data (e.g., including current and historic data). Other display devices can include other hand-held devices, such as display device 107d which represents a tablet, display device 107a which represents a smart watch or fitness tracker, medical device 108 (e.g., an insulin delivery device or a blood glucose meter), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end user, such as the patient) for each particular display device. Accordingly, in certain embodiments, a plurality of different display devices can be in direct wireless communication with a sensor electronics module (e.g., such as an on-skin sensor electronics module 138 that is physically connected to continuous analyte sensor(s) 140) during a sensor session to enable a plurality of different types and/or levels of display and/or functionality associated with the displayable sensor data.

As mentioned, sensor electronics module 138 may be in communication with a medical device 108. Medical device 108 may be a passive device in some example embodiments of the disclosure. For example, medical device 108 may be an insulin pump for administering insulin to a patient. For a variety of reasons, it may be desirable for such an insulin pump to receive and track lactate, glucose, ketones, glycerol and free fatty acid values transmitted from analyte sensor systems 104, where continuous analyte sensor 140 is configured to measure lactate, glucose, ketones, glycerol, and/or free fatty acids.

Further, as mentioned, sensor electronics module 138 may also be in communication with other non-analyte sensors 142. Non-analyte sensors 142 may include, but are not limited to, an altimeter sensor, an accelerometer sensor, a global positioning system (GPS) sensor, a temperature sensor, a respiration rate sensor, etc. Non-analyte sensors 142 may also include monitors such as heart rate monitors, blood pressure monitors, pulse oximeters, caloric intake monitors, indirect calorimetry devices, continuous positive airway pressure machines, and medicament delivery devices. One or more of these non-analyte sensors 142 may provide data to therapy management engine 112 described further below. In some aspects, a patient may manually provide some of the data for processing by the therapy management engine 112 of FIG. 1.

In certain embodiments, non-analyte sensors 142 may further include sensors for measuring movement or activity, blood pressure, skin temperature, core temperature, sweat rate, and/or sweat composition.

In certain embodiments, the non-analyte sensors 142 may be combined in any other configuration, such as, for example, combined with one or more continuous analyte sensors 140. As an illustrative example, a non-analyte sensor, e.g., a temperature sensor, may be combined with a continuous glucose sensor 140 to form a glucose/temperature sensor used to transmit sensor data to the sensor electronics module 138 using common communication circuitry. As another illustrative example, a non-analyte sensor, e.g., a temperature sensor, may be combined with a multi-analyte sensor 140 configured to measure lactate and glucose to form a lactate/glucose/temperature sensor used to transmit sensor data to the sensor electronics module 138 using common communication circuitry.

In certain embodiments, a wireless access point (WAP) may be used to couple one or more of analyte sensor system 104, the plurality of display devices, medical device(s) 108, and/or non-analyte sensor(s) 142 to one another. For example, such WAP may provide Wi-Fi and/or cellular connectivity among these devices. NFC and/or Bluetooth may also be used among devices depicted in diagram 150 of FIG. 1B.

FIG. 2 illustrates example inputs and example metrics that are generated based on the inputs in accordance with certain embodiments of the disclosure. In particular, FIG. 2 illustrates example inputs 127 on the left, application 106 and therapy management engine 112, with DAM 111, in the middle, and example outputs 130 on the right. In certain embodiments, application 106 may obtain inputs 127, in the form of time-series data, through one or more channels (e.g., continuous analyte sensor(s) 140, non-analyte sensor(s) 142, various applications executing on display device 107, etc.). Inputs 127 may be further processed by DAM 111 to output a plurality of metrics, such as outputs 130. Further, inputs (e.g., inputs 127) and metrics (e.g., outputs 130) may be used by the DAM 111 and/or any computing device in the system 100 to perform various processes. Any of inputs 127 may be used for computing any of outputs 130. In certain embodiments, each one of outputs 130 may correspond to one or more values, e.g., discrete numerical values, ranges, or qualitative values (high/medium/low or stable/unstable). In some embodiments, some or all of outputs 130 may include time-series data and/or be provided in the form of time-series data.

In certain embodiments, inputs 127 include food consumption information. Food consumption information may include information about one or more of meals, snacks, and/or beverages, such as one or more of the size, content (carbohydrate, fat, protein, etc.), sequence of consumption, and time of consumption. In certain embodiments, food consumption may be provided by the user through manual entry, by providing a photograph through an application that is configured to recognize food types and quantities, and/or by scanning a bar code or menu. In various examples, meal size may be manually entered as one or more of calories, quantity (e.g., ‘three cookies’), menu items (e.g., ‘Royale with Cheese’), and/or food exchanges (1 fruit, 1 dairy). In some examples, meals may also be entered with the user's typical items or combinations for this time or setting (e.g., workday breakfast at home, weekend brunch at restaurant). In some examples, meal information may be received via a convenient user interface provided by application 106.

In certain embodiments, inputs 127 include activity information. Activity information may be provided, for example, the one or more non-analyte sensors 142 of FIG. 1B. In certain embodiments, activity information may additionally be provided through manual input by user 102. Activity information may include, for example, a time series for each of heart rate, activity minutes, step count, floors climbed, location information (e.g., GPS data), calories burned, sleep duration and/or quality, activity level (e.g., light, medium, or heavy), and/or similar information. In addition, or alternatively, the activity information can include one or more time series for recorded activities of one or more defined activity types (e.g., walk, run, sprint, swim, weightlift etc.), where each activity is associated with a duration and/or time period.

In certain embodiments, inputs 127 include patient statistics, such as one or more of age, height, weight, body mass index, body composition (e.g., % body fat), stature, build, or other information. Patient statistics may be provided through a user interface, by interfacing with an electronic source such as an electronic medical record, and/or from measurement devices. The measurement devices may include one or more of a wireless, e.g., Bluetooth-enabled, weight scale and/or camera, which may, for example, communicate with the display device 107 to provide patient data.

In certain embodiments, inputs 127 include information relating to the user's medication intake. For example, the user's medication intake may include the user's insulin delivery. Such information may be received, via a wireless connection on a smart pen, via user input, and/or from an insulin pump (e.g., medical device 108). Insulin delivery information may include one or more of insulin volume, time of delivery, etc. Other configurations, such as insulin action time or duration of insulin action, may also be received as inputs.

In certain embodiments, inputs 127 include physiological information received from non-analyte sensor(s) 142, which may detect one or more of heart rate, respiration, oxygen saturation, body temperature, etc. (e.g., to detect illness, stress levels, etc.). In certain embodiments, inputs 127 include time, such as time of day, or time from a real-time clock.

In certain embodiments, inputs 127 include analyte data, which may be provided as input from analyte sensor system 104, for example, in any of the ways described with respect to FIG. 1A. An example of analyte data is glucose data, which may be provided and/or stored as a time series corresponding to time-stamped glucose measurements over time. Other types of analyte data, such as ketone data, potassium data, lactate data, etc., may similarly be provided and/or stored as a time series.

As described above, in certain embodiments, DAM 111 generates, determines, and/or computes outputs 130 based on inputs 127 associated with user 102. An example list of outputs 130 is illustrated in FIG. 2. In certain embodiments, outputs 130 generated, determined, or computed by DAM 111 include metabolic rate. Metabolic rate is a metric that may indicate or include a basal metabolic rate (e.g., energy consumed at rest) and/or an active metabolism, e.g., energy consumed by activity, such as exercise or exertion. In some examples, basal metabolic rate and active metabolism may be tracked as separate metric. In certain embodiments, the metabolic rate may be calculated by DAM 111 based on one or more of inputs 127, such as one or more of activity information, sensor input, time, user input, etc.

In certain embodiments, outputs 130 generated, determined, or computed by DAM 111 include an activity level metric. The activity level metric may indicate a level of activity of the user. In certain embodiments, the activity level metric may be determined, for example based on input from an activity sensor or other physiologic sensors. In certain embodiments, the activity level metric may be calculated by DAM 111 based on one or more of inputs 127, such as one or more of activity information, physiological information, analyte data, time, user input, etc. Activity level may indicate whether the user is exercising, at rest, sleeping, etc.

In certain embodiments, outputs 130 generated, determined, or computed by DAM 111 include an insulin resistance metric (also referred to herein as an “insulin resistance”). The insulin resistance metric may be determined using historical data, real-time data, or a combination thereof, and may, for example, be based upon one or more inputs 127, such as one or more of food consumption information, blood glucose information, insulin delivery information, the resulting glucose levels, etc. In certain embodiments, the insulin on board metric may be determined using insulin delivery information, and/or known or learned (e.g., from patient data) insulin time action profiles, which may account for both basal metabolic rate (e.g., update of insulin to maintain operation of the body) and insulin usage driven by activity or food consumption.

In certain embodiments, outputs 130 generated, determined, or computed by DAM 111 include a meal state metric. The meal state metric may indicate the state the user is in with respect to food consumption. For example, the meal state may indicate whether the user is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first.).

In certain embodiments, outputs 130 generated, determined, or computed by DAM 111 include health and sickness metrics. Health and sickness metrics may be determined, for example, based on one or more of user input (e.g., pregnancy information or known sickness information), from non-analyte sensor(s) 142, such as physiologic sensors (e.g., temperature), activity sensors, or a combination thereof. In certain embodiments, based on the values of the health and sickness metrics, for example, the user's state may be defined as being one or more of healthy, ill, rested, or exhausted. In certain embodiments, health and sickness metric may indicate the user's heart rate, stress level, etc.

In certain embodiments, outputs 130 generated, determined, or computed by DAM 111 include analyte level metrics. Analyte level metrics may be determined from analyte data (e.g., glucose measurements obtained from analyte sensor system 104). In some examples, an analyte level metric may also be determined, for example, based upon historical information about analyte levels in particular situations, e.g., given a combination of food consumption, insulin, and/or activity. An analyte level metric may include a rate of change of the analyte, time in range, time spent below a threshold level, time spent above a threshold level, or the like. In certain embodiments, an analyte trend may be determined based on the analyte level over a certain period of time. As described above, example analytes may include glucose, ketones, lactate, potassium and others described herein.

In certain embodiments, outputs 130 generated, determined, or computed by DAM 111 include a disease stage. For example disease stages for Type II diabetics may include a pre-diabetic stage, an oral treatment stage, and a basal insulin treatment stage. In certain embodiments, degree of glycemic control (not shown) may also be determined as an outcome metric, and may be based, for example, on one or more of glucose levels, variation in glucose level, or insulin dosing patterns.

In certain embodiments, outputs 130 generated, determined, or computed by DAM 111 include clinical metrics. Clinical metrics generally indicate a clinical state a user is in with respect to one or more conditions of the user, such as diabetes. For example, in the case of diabetes, clinical metrics may be determined based on glycemic measurements, including one or more of A1C, trends in A1C, time in range, time spent below a threshold level, time spent above a threshold level, and/or other metrics derived from glucose values. In certain embodiments, clinical metrics may also include one or more of estimated A1C, glycemic variability, hypoglycemia, and/or health indicator (time magnitude out of target zone).

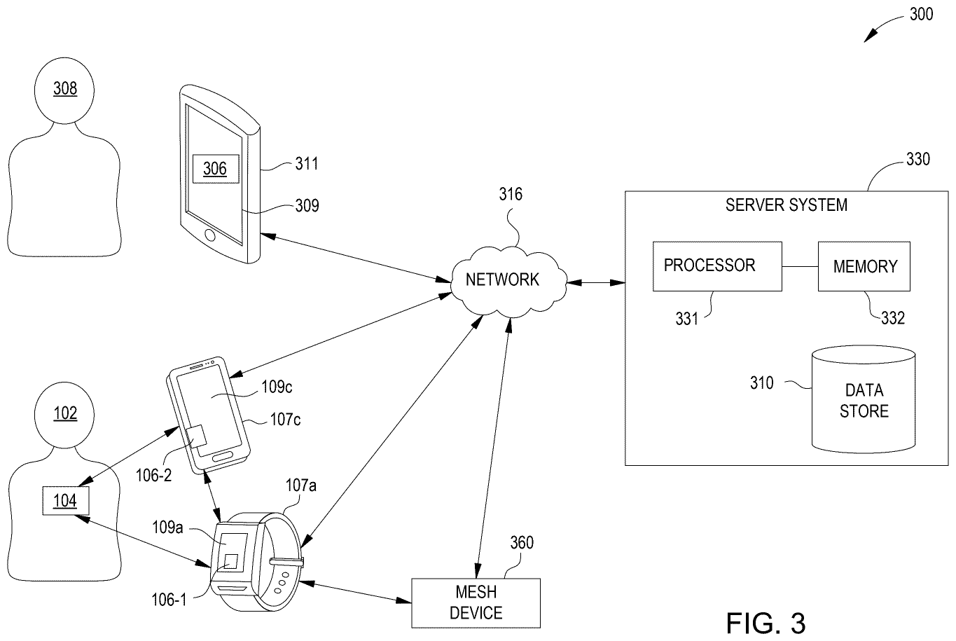

FIG. 3 illustrates an example of a system 300 for sharing a patient's health data between devices and/or with one or more other users (referred to herein as supporters), in accordance with certain embodiments. System 300 includes the analyte sensor system 104 of FIGS. 1A-B, the display devices 107a and 107c of FIG. 1B, a supporter device 311, and a server system 330. The display devices 107a and 107c, the supporter device 311, and the server system 330 are interconnected via a network 316. The network 316 can be a local area network (LAN), wide area network (WAN), a cellular network, the Internet, or any other group of connected computing devices.

The user 102 can use one or both of the display devices 107a and 107c to collect sensor data and monitor their health. More particularly, in the illustration of FIG. 3, the display devices 107a and 107c are examples of multiple receiver devices, namely, a smartphone and a smartwatch, that each directly communicate with, and obtain sensor data from, the analyte sensor system 104 worn by the user 102, where such sensor data can be transmitted by the analyte sensor system 104 in the fashion described relative to FIGS. 1A-B. According to the example of FIG. 3, the display devices 107a and 107c may each be included in the connection list discussed relative to the transceiver 136 of FIG. 1B. Although two receiver devices are shown in FIG. 3, it should be appreciated that, in various implementations, any number of devices may directly receive such sensor data including, for example, any one or more of display devices 107a, 107b, 107c, and 107d of FIG. 1B. Display devices 107a and 107c may include touchscreen displays 109a and 109c, respectively, as discussed relative to FIG. 1B. As shown, display devices 107a and 107c execute application 106-1 and application 106-2, respectively, which applications each correspond to instances of the application 106 discussed relative to FIGS. 1A-B and 2.

In certain aspects, a supporter 308 uses the supporter device 311 to help the user 102 manage their health or medical condition, such as diabetes. The supporter device 311 can execute an application 306, which can access at least selected health data of the user 102 (e.g., analyte data and/or alerts related thereto) from the server system 330 and can display the health data via a display 309 of the supporter device 311. In some aspects, the application 306 can operate as described relative to the application 106 of FIG. 1. Although the supporter 308 and the supporter device 311 are shown singly in FIG. 3, it should be appreciated that, in various implementations, the supporter 308 and/or the supporter device 311 may be representative of multiple supporters and/or supporter devices, respectively.

In the example of FIG. 3, the server system 330 is an example of a remote target that facilitates, for example, publishing or providing health data of the user 102 to the supporter 308. In some aspects, the server system 330 is operable in a cloud computing environment (e.g., a private or public cloud) and can provide application services and/or features for the analyte sensor system 104 and/or applications 106-1, 106-2, and/or 306. In certain aspects, the server system 330 implements at least a portion of the therapy management engine 112 described with respect to FIG. 1A. The server system 330 can receive and store data generated by the analyte sensor system 104 (via transmissions initiated by the applications 106-1 and/or 106-2), monitor operating parameters of the analyte sensor system 104 (via the applications 106-1 and/or 106-2), publish or provide health data to the supporter device 311, push updates to the applications 106-1, 106-2 and/or 306, etc.

The server system 330 includes a processor 331 configured to execute one or more software applications stored in a memory 332. For example, the processor 331 can execute a data sharing application generally configured to enable the supporter 308 to receive health data of the user 102 and monitor the health of the user 102. For example, in some aspects, once the user 102 opts into receiving services provided by the server system 330, the server system 330 is able to receive the health data transmitted by the display devices 107a and/or 107c and to store the health data in a storage system (e.g., data store 310). Once the user 102 opts into allowing the supporter 308 to receive information related to the health of the user 102, the server system 330 can allow the supporter 308 to also access the health data within the storage system.

In general, to enable publication of health data of the user 102 to the supporter 308, the analyte sensor system 104 may be configured to generate time-series sensor data, such as analyte measurements, for the user 102, e.g., on a continuous basis. The analyte sensor system 104 transmits the sensor data to each receiver device connected thereto (e.g., any of the display devices 107) via, for example, the transceiver 136 discussed relative to FIG. 1B. For example, the analyte sensor system 104 can transmit the sensor data to each device included in the connection list discussed relative to the transceiver 136 of FIG. 1B. In the example of FIG. 3, the sensor data may be transmitted to each of the display devices 107a and 107c for use by the application 106-1 and the application 106-2, respectively.

In certain aspects, one or both of the display device 107a and the display device 107c may transmit the sensor data to the server system 330. The server system 330 can forward the sensor data and/or alerts or other health data based on the sensor data to the supporter device 311, thereby allowing the supporter 308 to view the patient's analyte data and/or alerts substantially in real-time. In some aspects, any resulting redundant transmissions to the server system 330 can be de-duplicated upon receipt of the sensor data, upon transmission of alerts, for example, to the supporter device 311, and/or the like. The redundant transmissions can be de-duplicated, for example, based on timestamps, a combination of timestamps and data content, and/or the like.

In the example of FIG. 3, the display devices 107a and 107c are configured to directly communicate with the analyte sensor system 104, as discussed above, and also to directly communicate with each other. In particular, communication paths are established between the analyte sensor system 104 and the display device 107a, between the analyte sensor system 104 and the display device 107c, and between the display device 107a and the display device 107c. In certain aspects, communication between the display device 107a and the display device 107c, for example, can occur over the network 316. In addition, or alternatively, such communication can occur directly between the two devices without the network 316.

In certain aspects, the display devices 107a and 107c are configured to optimize presentation of alerts across the two devices, for example, based on analyte data from the analyte sensor system 104. Although, for simplicity of description, certain illustrative examples are described below in terms of two devices and an analyte sensor system, it should appreciated that similar optimization functionality can be achieved with three, four, five, six, or any other suitable number devices that are each operable to communicate with an analyte sensor and each other. For example, and not by way of limitation, similar functionality may be performed relative to any combination of the display devices 107a, 107b, 107c, and 107d of FIG. 1B.

In certain aspects, the display devices 107a and 107c can each be configured to optimize presentation of alerts across the devices by performing synchronized alert de-duplication. For example, in certain aspects, the applications 106-1 and 106-2 can each be configured to independently execute alert de-duplication prior to presenting an alert at the display device 107a and 107c, respectively.

For each alert, an alert identifier can be deterministically generated, such that the same identifier is generated for all alerts that result from the same trigger, regardless of where (e.g., at which device) the alert is generated. For example, the alert identifier can be a combination of an alert type (e.g., high, low, etc.) and a time associated with an analyte measurement that resulted in the alert. The time associated with the analyte measurement can be, for example, a timestamp indicative of date and time, a sensor tick time (e.g., a time value that increments from when the analyte sensor system 104 is first instantiated), or the like. In addition, or alternatively, the alert identifier can include information to distinguish the alert by sensor data type (e.g., for implementations involving multiple sensors such as glucose, lactate, ketone and/or the like). For example, the alert identifier can include an identifier of a sensor type, a hash of analyte data resulting in the alert, and/or the like. In various aspects, the display devices 107a and 107c may each share alerts generated thereon with the other device, for example, by sending alert notifications that include the alert identifiers.

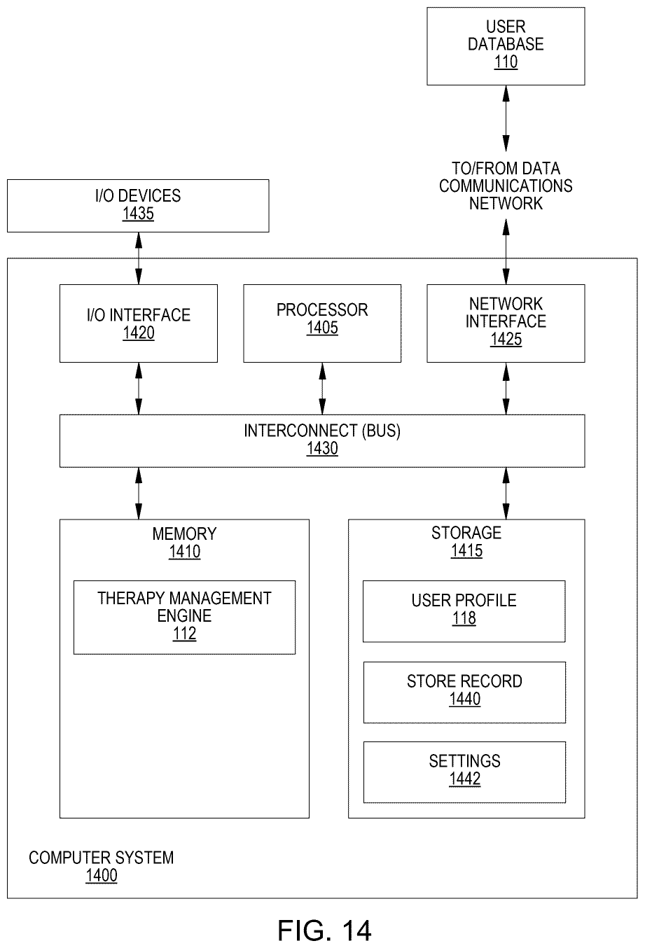

In certain aspects, the display devices 107a and 107c can each maintain in memory thereon, or have access to (e.g., over the network 316), a stored record of historical alerts previously processed at the device, for example, due to generation at that device or to receipt of an alert notification from another device (e.g., shown as stored record 1440 in FIG. 14). For each alert, the stored record can include, for example, an alert identifier, whether the alert was presented, time(s) of presentation of the alert (if applicable), a source of the alert (e.g., a determination at that device or a notification from another device), an alert status (e.g., whether the alert has been acknowledged or responded to with treatment), and/or other suitable information. In some aspects, to achieve memory savings, the stored record can include a listing of alert identifiers to the exclusion of other information, such as any of the information listed above. For each of the display devices 107a and 107c, the stored record can be maintained in the device's memory or in another storage location accessible to the device.

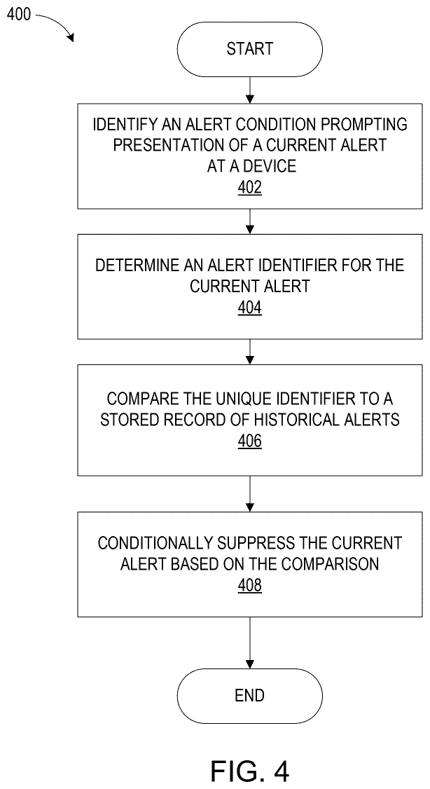

In certain aspects, for each of the display devices 107a and 107c, the stored record can be updated with a corresponding alert identifier and/or other data each time an alert is processed at the device. In certain aspects, as part of the alerting process, the display devices 107a and 107c can each conditionally suppress a current alert (e.g., cause the current alert to be presented or suppressed) based on whether an alert with the same alert identifier has already been presented on that device (e.g., based on a comparison with the stored record of historical alerts previously processed at the device). In this way, the display devices 107a and 107c can each independently execute alert deduplication in response to an alert generated at the device and/or in response to an alert notification received from another receiver device. Examples of alert deduplication will be described in greater detail relative to FIGS. 4-6.

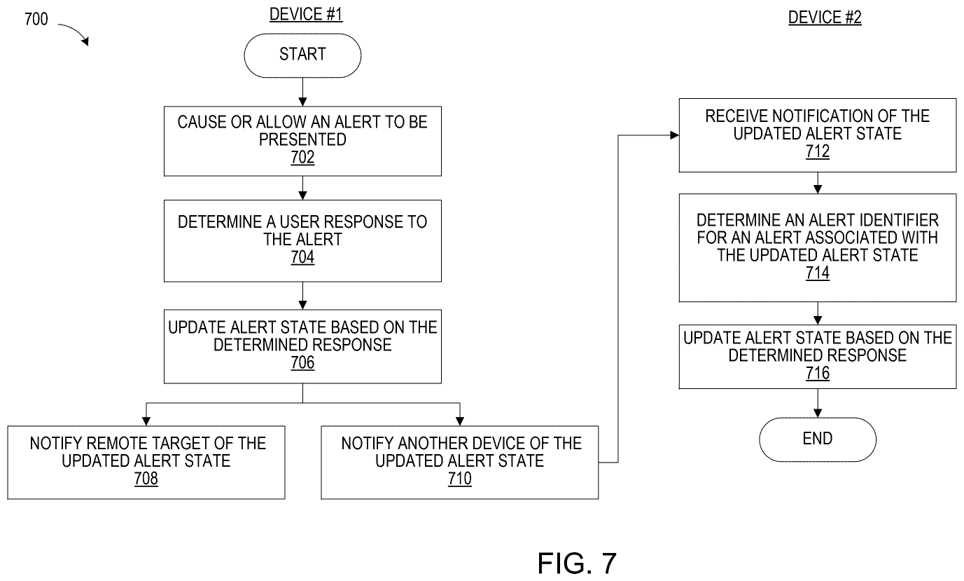

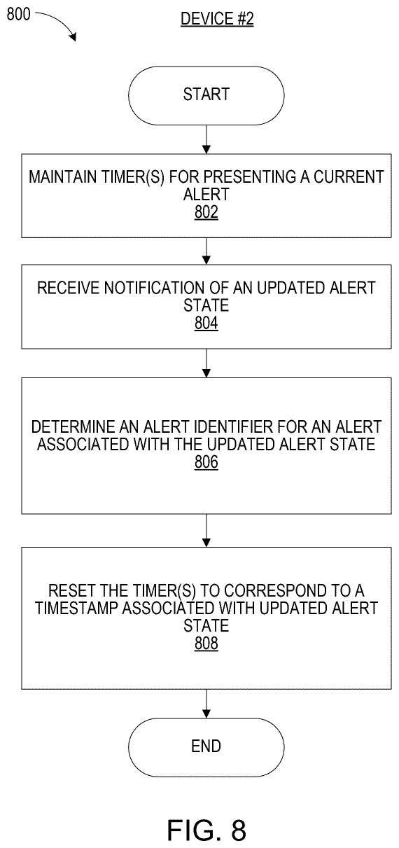

In certain aspects, the display devices 107a and 107c can each be configured to optimize presentation of alerts across the devices by synchronizing alerts states. For example, in certain aspects, the applications 106-1 and 106-2 can each be configured to execute one or more processes to share updates to alert states with other receiver devices, such as the display devices 107c and 107a, respectively. The updates can include or indicate, for example, a user response to a given alert, such as whether the alert has been acknowledged by the user (e.g., via a button press or swipe), whether the alert has been responded to with treatment, and/or the like. For example, the display device 107c can determine an updated alert state (e.g., that an alert has been acknowledged) and can share the updated alert state with the display device 107a. The updated alert state can include, for example, an alert identifier, as discussed above, and a timestamp (e.g., a time of acknowledgement). In response, the display device 107a can suppress the alert at that device, clear the alert at that device, adjust timing for re-presentation of the alert at that device, and/or the like. Examples of sharing updates to alert state, and taking automated action in response thereto, will be described in greater detail relative to FIGS. 7-8.

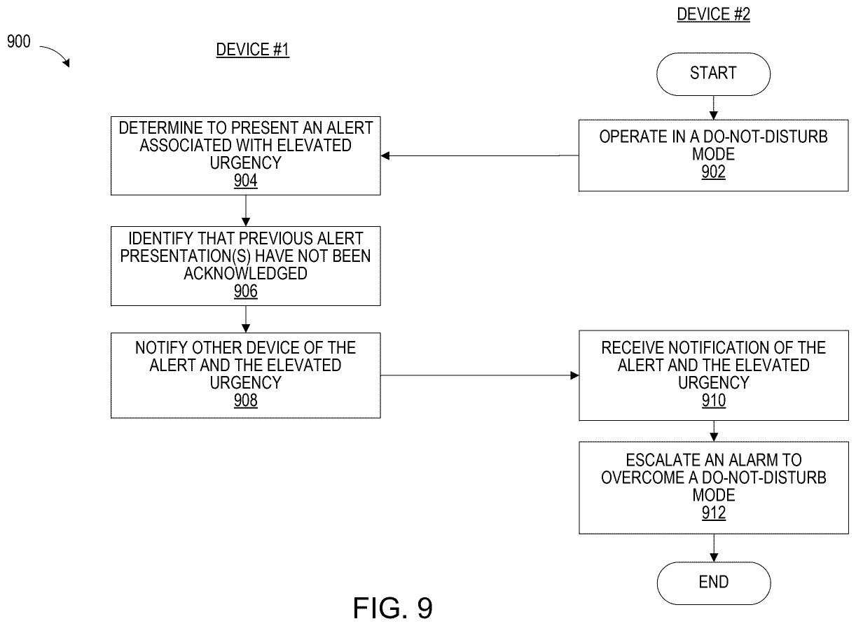

In some aspects, the sharing of alert states can be adapted or optimized in response to device states or modes that impact alert presentation. In an example, if the display device 107a is in a DND mode, or a similar mode that prevents alert presentation, and configurable conditions are present (e.g., the situation is urgent and/or alerts are not being acknowledged at the display device 107c), the display device 107c can indicate the elevated urgency to the display device 107a, thereby causing the display device 107a to escalate an alarm to overcome the DND mode and present the alert thereon. Examples of overcoming, for example, DND mode, will be described in greater detail relative FIG. 9.

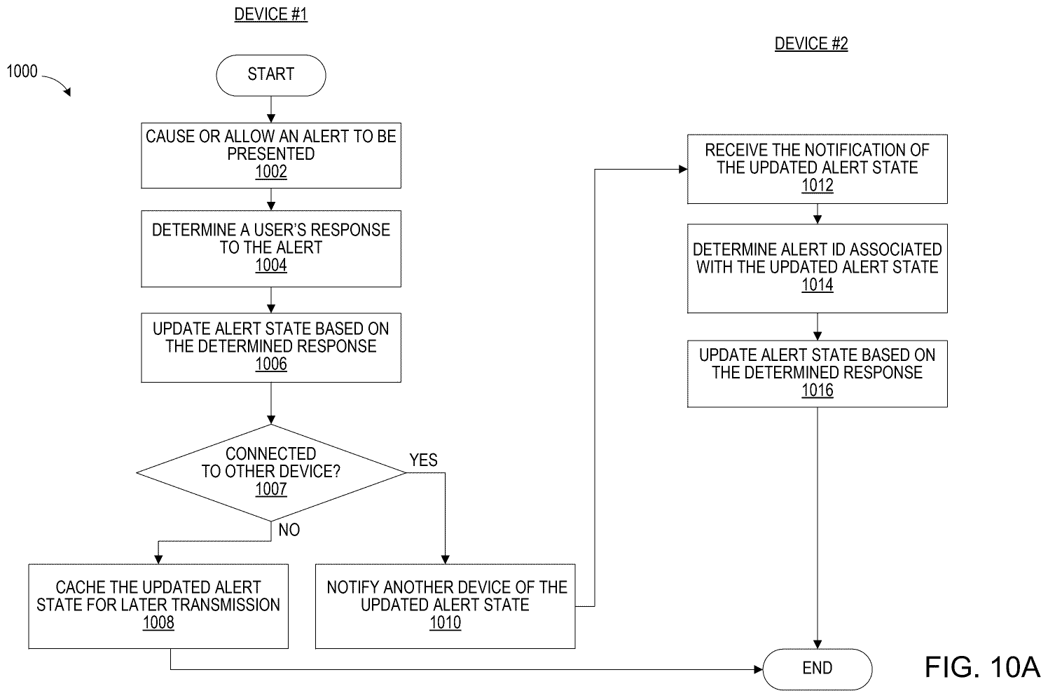

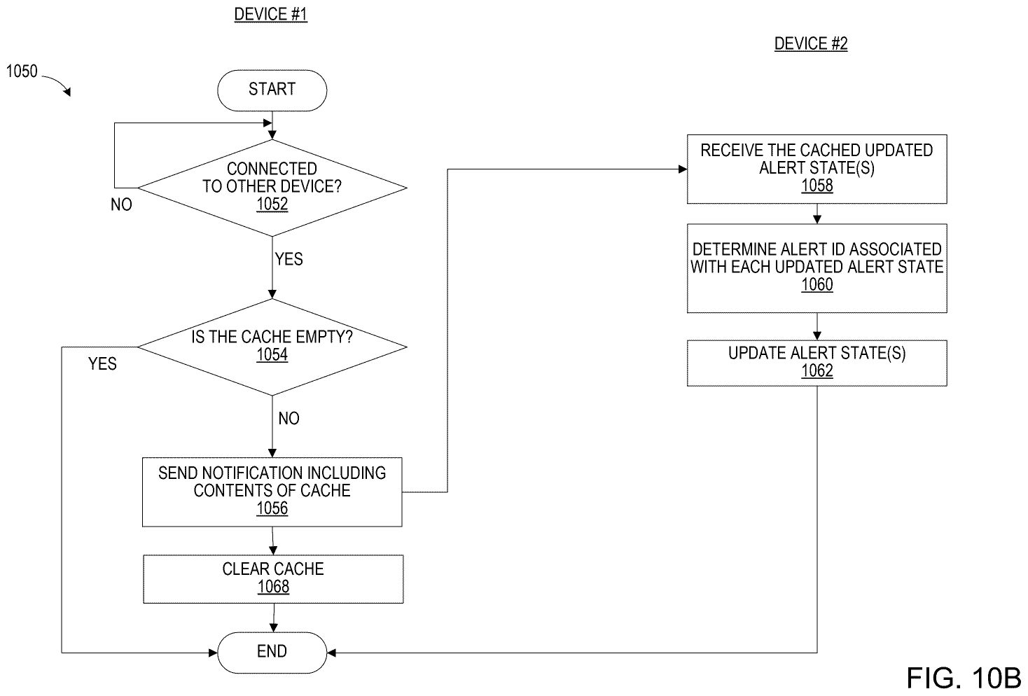

In another example of adapting or optimizing the sharing of alert states, if communication between the display device 107a and the display device 107c is interrupted, the display device 107c can cache an updated alert state for later transmission. The display device 107c can then transmit the updated alert state to the display device 107a, for example, upon reestablishment of a connection therewith. Examples of caching an updated alert state for later transmission will be described in greater detail relative to FIGS. 10A-B.

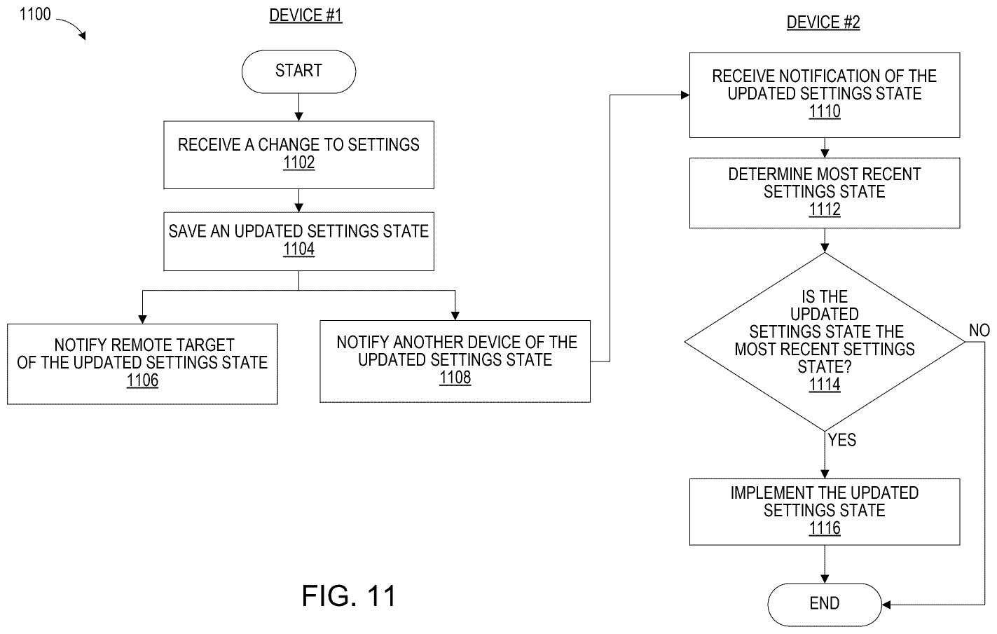

In certain aspects, the display devices 107a and 107c can each be configured to optimize presentation of alerts across the devices by synchronizing settings. The display devices 107a and 107c can each maintain a set of settings (e.g., shown as settings 1442 in FIG. 14). For example, in certain aspects, the applications 106-1 and 106-2 can each be configured to execute one or more processes to share changes to settings with other receiver devices, such as the display device 107c and 107a, respectively. In certain aspects, the display devices 107a and 107c can synchronize settings by maintaining settings states in relation to time stamps. When settings are changed at the display device 107a, for example, the display device 107a can save an updated settings state in association with a timestamp of the change. The updated settings state and associated time stamp can be shared with other devices, such as the display device 107c, thereby enabling synchronization of settings between multiple devices. In various cases, the display devices 107a and 107c can each determine and implement a most recent settings state based on timestamp. In addition, or alternatively, the display devices 107a and 107 can each roll back to an earlier settings state based on similar timestamps. Examples of synchronizing settings will be described in greater detail relative to FIG. 11.

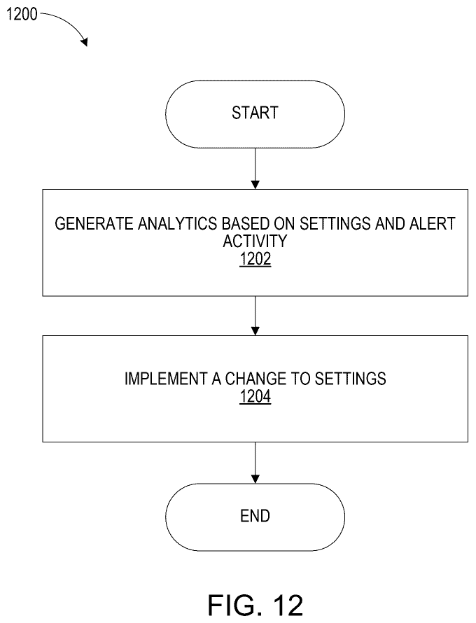

In certain aspects, the display devices 107a and 107c can each be configured to optimize presentation of alerts by generating alert analytics, for example, based on the stored record of historical alerts on or accessible to each device. In some aspects, the analytics may be used to perform, for example, personalized optimization of settings to decrease alert fatigue and increase user engagement with alerts. In some aspects, the analytics may be used to dynamically establish or change a priority device for alert presentation based on user behavior (e.g., based on time and/or location). Examples of generating analytics based on settings and alert activity will be described in greater detail relative to FIG. 12.

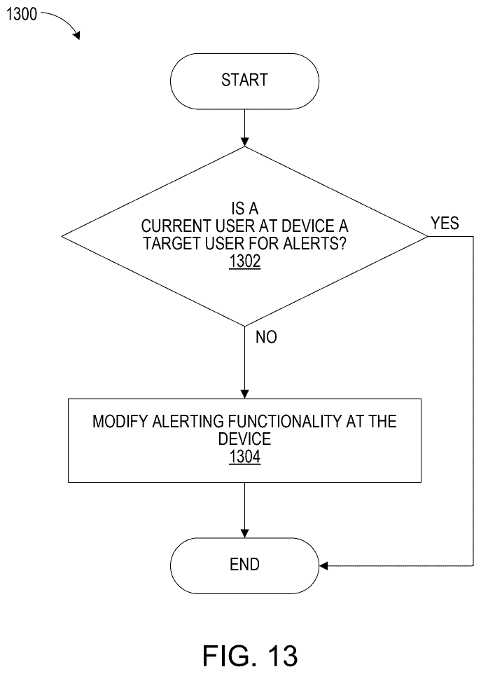

In certain aspects, the display devices 107a and 107c can each be configured to optimize presentation of alerts by adapting settings to a current user thereof. For example, if the display device 107c determines that it is currently being used by a non-target user (e.g., someone other than the user 102), such as a child of the user 102, the display device 107c can temporarily modify alerting functionality, for example, by overriding any of the aforementioned alert suppression features, implementing additional alert sharing, etc. For example, acknowledgements and/or other alert state updates at the display device 107c may be ignored, not recorded, and/or not substantively acted upon. In this way, when the display device 107c is returned to the user 102, previous alerts may still be viewable and/or re-presentation of alerts may still occur. In addition, or alternatively, alert state updates may not be shared with other devices, such as the display device 107a. In addition, or alternatively, any suppression of sharing of alerts with other devices, such as the display device 107a, may be overridden (e.g., turned off). Examples of adapting settings to a current user will be described in greater detail relative to FIG. 13.

FIG. 4 illustrates an example of a process 400 for executing alert deduplication at a device, in accordance with certain embodiments. In some embodiments, the process 400 can be executed, for example, by the therapy management engine 112 of FIGS. 1A-B and 2. In addition, or alternatively, the process 400 can be executed, for example, by the application 106 of FIGS. 1A-B and 2. In addition, or alternatively, the process 400 can be executed generally by any of the display devices 107 of FIGS. 1A-B and 2.

Although any number of systems, in whole or in part, can implement the process 400, to simplify discussion, the process 400 will be described as being performed by the application 106 of FIGS. 1A-B and 2, with particular reference to the system 300 of FIG. 3. With reference to the system 300 of FIG. 3, in some aspects, the process 400 can be executed continuously and in parallel by each of the applications 106-1 and 106-2. In addition, or alternatively, the process 400 can be executed by the application 106-1 and the application 106-2 upon the occurrence or determination of an event or condition, such as receipt of analyte data from the analyte sensor system 104. Other variations will be apparent to one skilled in the art after a detailed review of the present disclosure.

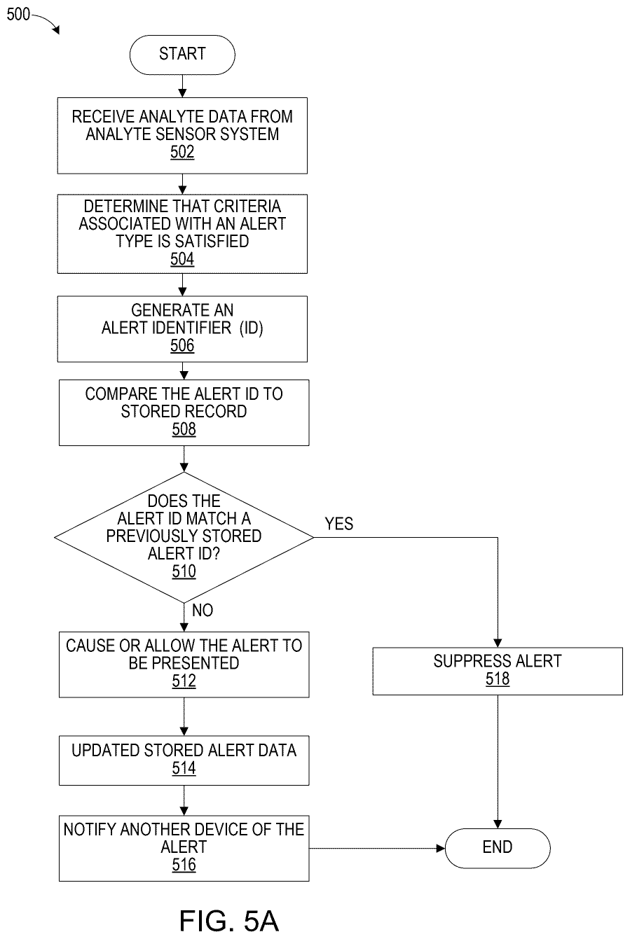

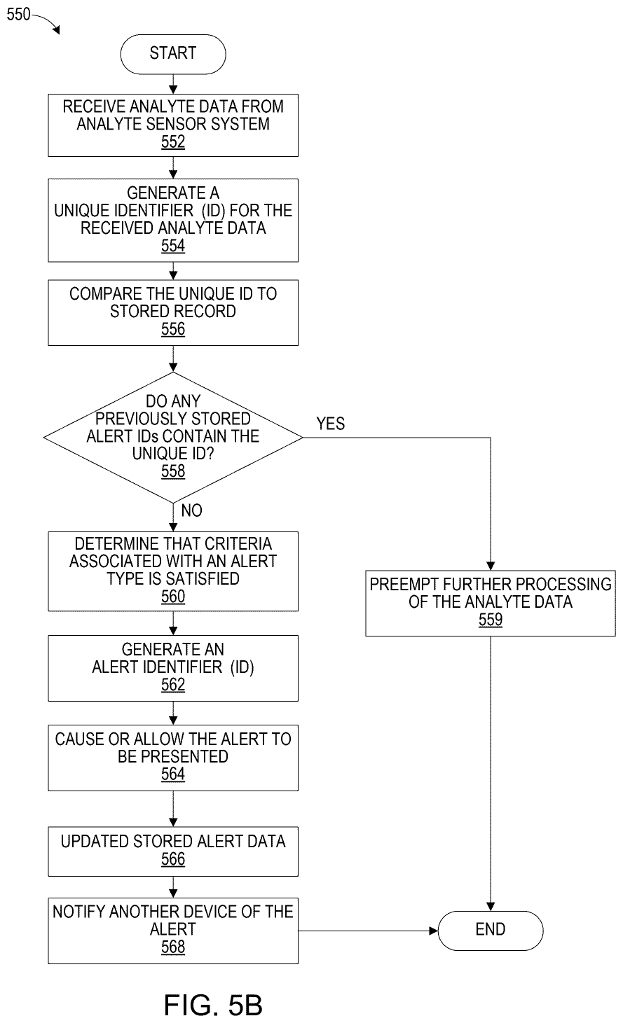

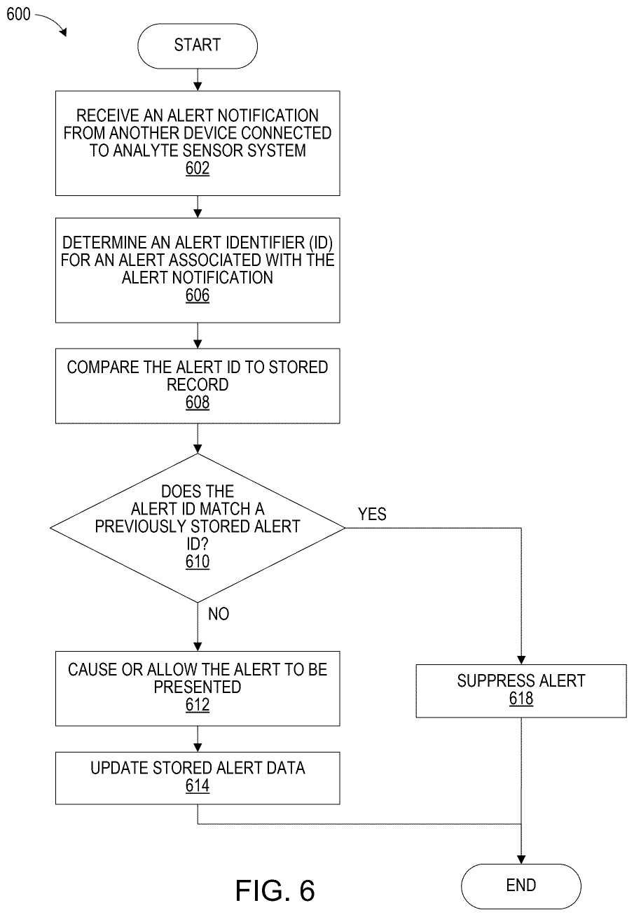

At block 402, the application 106 identifies an alert condition prompting presentation of a current alert at the display device 107. In an example, the alert condition can be a determination at the display device 107 based on analyte measurements received directly from the analyte sensor system 104, such as a determination that the analyte measurements satisfy criteria associated with a type of the current alert (e.g., a high alert associated with a high threshold, a low alert associated with a low threshold, etc.). In another example, the alert condition can be a notification from another device that the current alert has been generated or presented at that other device, for example, based on analyte measurements that the other device received. Examples of the alert condition being a determination at the display device 107 will be discussed relative to FIGS. 5A-B. An example of the alert condition being a notification from another device will be discussed relative to FIG. 6.

At block 404, the application 106 determines an alert identifier for the current alert. The alert identifier can be, for example, a unique identifier that distinguishes the current alert from other alerts based on a trigger for the alert. In certain aspects, each alert can have a deterministically generated alert identifier, such that the same identifier is generated for all alerts that result from the same trigger, regardless of where (e.g., at which device) the alert is generated. For example, the alert identifier may be a combination of an alert type (e.g., high, low, etc.) and a time associated with an analyte measurement that resulted in the alert (e.g., a timestamp indicative of date and time, a sensor tick time that increments from when the analyte sensor system 104 is first instantiated, etc.). In addition, or alternatively, the alert identifier can include information to distinguish the alert by sensor data type (e.g., for implementations involving multiple sensors such as glucose, lactate, ketone and/or the like). For example, the alert identifier can include an identifier of a sensor type, a hash of analyte data resulting in the alert, and/or the like. In cases where the alert condition for the current alert is a determination at the display device 107, the block 404 can include, for example, generating the alert identifier, as will be discussed in greater detail relative FIGS. 5A-B. In cases where the alert condition for the current alert is a notification from another device, the block 404 can include, for example, determining the alert identifier from the notification, as will be discussed in greater detail relative to FIG. 6.

At block 406, the application 106 automatically compares the alert identifier for the current alert to a stored record of historical alerts previously processed at the display device 107. As discussed relative to FIG. 3, in certain aspects, the display device 107 can maintain the stored record in its memory, or can otherwise have access to the stored record, for example, over the network 316. As discussed previously, the historical alerts may have been previously processed at the display device 107, for example, due to generation at the display device 107 or to receipt of alert notifications from one or more other devices. The block 406 can include, for example, comparing the alert identifier for the current alert to alert identifiers for the historical alerts of the stored record. In general, the existence of the alert identifier in the stored record can signify that the display device 107 has already presented the same alert.

At block 408, the application 106 conditionally suppresses the current alert on the display device 107 based on the comparison at the block 406. In an example, if the alert identifier for the current alert matches an alert identifier for any of the historical alerts of the stored record, the application 106 can suppress the current alert at the display device 107 (e.g., prevent the current alert from being presented at the display device 107), thereby avoiding presentation of a redundant alert. In general, the suppression can include the application 106 taking any suitable action or non-action to prevent the alert from being presented at the display device 107. The suppression can include, for example, discarding the alert, recording the redundant alert in the stored record without taking further action, incrementing a counter associated with the alert identifier in the stored record, not initiating or causing presentation according to a typical alerting flow, combinations of the foregoing and/or the like. For example, the suppression can include the application 106 skipping execution of a software routine to further process the current alert and/or any data related thereto, thereby saving processing resources at the display device 107.

In another example, if the alert identifier for the current alert does not match an alert identifier for any of the historical alerts of the stored record, at the block 408, the application 106 can cause or allow presentation of the current alert at the display device 107, for example, via any suitable visual, audio and/or haptic feedback. Examples of conditional suppression will be described in greater detail relative to FIGS. 5A-B and 6. After block 408, the process 400 ends.

FIG. 5A illustrates an example of a process 500 for executing alert deduplication in response to an alert determination at a device, in accordance with certain embodiments. In some embodiments, the process 500 can be executed, for example, by the therapy management engine 112 of FIGS. 1A-B and 2. In addition, or alternatively, the process 500 can be executed, for example, by the application 106 of FIGS. 1A-B and 2. In addition, or alternatively, the process 500 can be executed generally by any of the display devices 107 of FIGS. 1A-B and 2.

Although any number of systems, in whole or in part, can implement the process 500, to simplify discussion, the process 500 will be described as being performed by the application 106 of FIGS. 1A-B and 2, with particular reference to the system 300 of FIG. 3. With reference to the system 300 of FIG. 3, in some aspects, the process 500 can be executed continuously and in parallel by each of the applications 106-1 and 106-2. In addition, or alternatively, the process 500 can be executed by the application 106-1 and the application 106-2 upon the occurrence or determination of an event or condition, such as receipt of analyte data or an alert from the analyte sensor system 104 or another device or system. Other variations will be apparent to one skilled in the art after a detailed review of the present disclosure.

At block 502, the application 106 receives analyte data (e.g., analyte measurements) from the analyte sensor system 104. The analyte data may be received, for example, via the transceiver 136 of the analyte sensor system 104, as discussed relative to FIGS. 1A-B and 3.

At block 504, the application 106 determines that criteria associated with an alert type is satisfied (e.g., high, low, rapidly increasing, rapidly decreasing, etc.). In certain aspects, the determination at the block 504 corresponds to an example of an alert condition to present a current analyte alert, as discussed relative to the block 402 of FIG. 4. The block 504 can include, for example, determining that the analyte measurements received at the block 502 satisfy the criteria associated with the alert type (e.g., one or more thresholds).

At block 506, the application 106 generates an alert identifier for an alert of the alert type. In certain aspects, the block 506 may occur as part of determining an alert identifier for a current analyte alert, as discussed relative to the block 404 of FIG. 4. The alert identifier can be, for example, a key or other unique identifier that is generated based on the alert type and a trigger for the alert. The trigger may be, for example, the analyte data that resulted in the alert. The alert identifier can be, for example, a value or combination of values that is deemed unique with respect to the trigger for the alert. As discussed previously, in certain aspects, the alert identifier can be deterministically generated such that, for any two or more devices generating an alert of the same type based on the same analyte data, the same alert identifier would be generated by each device.

For example, the alert identifier may be a combination (e.g., concatenation) of an alert type identifier and a time associated with an analyte measurement that resulted in the alert. The alert type identifier can be a predetermined value indicative of the alert type, such as a high alert, a low alert, a rapidly increasing alert, a rapidly decreasing alert, etc. The time can be a time associated with the analyte measurement that generated the alert, such as a timestamp indicative of date and time, a sensor tick time (e.g., a time value that increments from when the analyte sensor system 104 is first instantiated) or the like, thereby ensuring a consistent reference point across different devices. The alert identifier can also be deterministically generated using other data values and/or in other suitable ways. For example, in some aspects, the alert identifier can be generated as a hash of a suitable combination of data values related to the alert.

At block 508, the application 106 automatically compares the alert identifier generated at the block 506 to a stored record of historical alerts previously processed at the display device 107. In general, the block 508 can include performing any of the functionality discussed relative to the block 406 of FIG. 4.

At decision block 510, the application 106 determines whether the alert identifier for the alert matches an alert identifier for any of the historical alerts of the stored record. As discussed previously relative to FIG. 4, the existence of the alert identifier in the stored record can signify that the display device 107 has already presented the same alert. The display device 107 may have already presented the same alert, for example, as a result of having previously received an alert notification from another device, as will be discussed relative to FIG. 6. In certain aspects, blocks 510, 512, and 518 together serve as an example of conditional suppression as discussed relative to the block 408 of FIG. 4.

If it is determined, at the decision block 510, that the alert identifier for the alert matches an alert identifier for a historical alert of the stored record, at block 518, the application 106 suppresses the alert, thereby avoiding presentation of a redundant alert. In general, the suppression can include any suitable action or non-action to prevent the alert from being presented at the display device 107. The suppression can include, for example, discarding the alert, recording the redundant alert in the stored record without taking further action, incrementing a counter associated with the alert identifier in the stored record, not initiating or causing presentation according to a typical alerting flow, combinations of the foregoing and/or the like.

If it is determined, at the decision block 510, that the alert identifier does not match an alert identifier for any historical alert of the stored record, at block 512, the application 106 causes or allows the alert to be presented, for example, via any suitable visual, audio and/or haptic feedback. At block 514, the application 106 updates the stored record of historical alerts to include the alert identifier, for example, by adding the alert identifier to the stored record. In this way, future presentations of alerts having the same alert identifier can be conditionally suppressed.

At block 516, the application 106 notifies another receiver device of the alert, for example, by sending an alert notification to the other device. In an example, if the application 106 corresponds to the application 106-1 of FIG. 3, the application 106-1 can cause the display device 107a to notify the display device 107c. In another example, if the application 106 corresponds to the application 106-2 of FIG. 3, the application 106-2 can cause the display device 107c to notify the display device 107a. The alert notification can include, for example, the alert identifier, so that the other device can execute alert logic to determine whether to present the alert, as will be discussed relative to FIG. 6. The alert notification can include, for example, the alert identifier and other data about the alert, such as information related to a time associated with the alert, analyte data that triggered the alert, and/or the like. In some aspects, as a performance optimization, the alert notification can include the alert identifier and exclude other data about the alert, thereby preserving network bandwidth. After either block 516 or block 518, the process 500 ends.

FIG. 5B illustrates an example of a process 550 for preventing redundant processing of analyte data, in accordance with certain embodiments. In some embodiments, the process 550 can be executed, for example, by the therapy management engine 112 of FIGS. 1A-B and 2. In addition, or alternatively, the process 550 can be executed, for example, by the application 106 of FIGS. 1A-B and 2. In addition, or alternatively, the process 550 can be executed generally by any of the display devices 107 of FIGS. 1A-B and 2.

Although any number of systems, in whole or in part, can implement the process 550, to simplify discussion, the process 550 will be described as being performed by the application 106 of FIGS. 1A-B and 2, with particular reference to the system 300 of FIG. 3. With reference to the system 300 of FIG. 3, in some aspects, the process 550 can be executed continuously and in parallel by each of the applications 106-1 and 106-2. In addition, or alternatively, the process 550 can be executed by the application 106-1 and the application 106-2 upon the occurrence or determination of an event or condition, such as receipt of analyte data or an alert from the analyte sensor system 104 or another device or system. Other variations will be apparent to one skilled in the art after a detailed review of the present disclosure.

At block 552, the application 106 receives analyte data (e.g., analyte measurements) from the analyte sensor system 104. The analyte data may be received, for example, via the transceiver 136 of the analyte sensor system 104, as discussed relative to FIGS. 1A-B and 3.

At block 554, the application 106 generates a unique identifier for the received analyte data. The unique identifier is distinct from an alert identifier, as discussed above relative to FIG. 5A, since, at the time block 554 is executed, the application 106 has not yet determined whether the analyte data would be a trigger for an alert. However, the unique identifier can be generated in the same way that at least a portion of an alert identifier would be generated, for example, if the received analyte data were to be a trigger for an alert. Stated somewhat differently, the unique identifier may be based on one or more of the same data values on which alert identifiers are based. In this way, as further discussed below relative to block 556, the unique identifier can be efficiently compared with corresponding portions of alert identifiers, even though no alert is in view at the block 554.

For example, the unique identifier can be derived from a time associated with the received analyte data. The time can be a time associated with an analyte measurement, such as a timestamp indicative of local date and time that is synchronized across devices, a sensor tick time (e.g., a time value that increments from when the analyte sensor system 104 is first instantiated) or the like, thereby ensuring a consistent reference point across different devices. The unique identifier can also be deterministically generated using other data values and/or in other suitable ways. For example, in some aspects, the unique identifier can be generated as a hash of a suitable combination of data values related to the analyte data.

At block 556, the application 106 automatically compares the unique identifier generated at the block 554 to a stored record of historical alerts previously processed at the display device 107. As discussed relative to FIG. 3, in certain aspects, the display device 107 can maintain the stored record in its memory, or can otherwise have access to the stored record, for example, over the network 316. As discussed previously, the historical alerts may have been previously processed at the display device 107, for example, due to generation at the display device 107 or to receipt of alert notifications from one or more other devices. The block 556 can include, for example, comparing the unique identifier to alert identifiers for the historical alerts of the stored record. As discussed previously, at least a portion of the alert identifiers can be of a same data type and/or format as the unique identifier, thus facilitating efficient comparison.

At decision block 558, the application 106 determines whether any of the previously stored alert identifiers contain the unique identifier for the received analyte data. In general, the fact that an alert identifier contains the unique identifier can signify that the display device 107 has already presented an alert based on the received analyte data. The display device 107 may have already presented an alert, for example, as a result of having previously received an alert notification from another device, as will be discussed relative to FIG. 6.

If it is determined, at the decision block 558, that at least one previously stored alert identifier contains the unique identifier for the received analyte data, at block 559, the application 106 preempts further processing of the received analyte data. In general, the preemption can include, for example, termination of the process 550. In addition, or alternatively, the preemption can include, for example, discarding the analyte data, recording the redundant receipt of the analyte data in the stored record without taking further action, incrementing a counter associated with the corresponding alert identifier in the stored record, combinations of the foregoing and/or the like. Advantageously, in certain aspects, the preemption at the block 559 can save processing resources at the display device 107 and preserve battery capacity at the display device 107.

If it is determined, at the decision block 558, that none of the previously stored alert identifiers contains the unique identifier for the received analyte data, the process 500 proceeds to block 560. At block 560, the application 106 determines that criteria associated with an alert type is satisfied (e.g., high, low, rapidly increasing, rapidly decreasing, etc.), as discussed relative to block 504 of FIG. 5A. At block 562, the application 106 generates an alert identifier for an alert of the alert type, as discussed relative to block 506 of FIG. 5A.

At block 564, the application 106 causes or allows the alert to be presented, for example, via any suitable visual, audio and/or haptic feedback, as discussed relative to block 512 of FIG. 5A. At block 566, the application 106 updates the stored record of historical alerts to include the alert identifier, for example, by adding the alert identifier to the stored record, as discussed relative to block 514 of FIG. 5A. At block 568, the application 106 notifies another receiver device of the alert, for example, by sending an alert notification to the other device, as discussed relative to block 516 of FIG. 5A. After either block 559 or block 568, the process 550 ends.

FIG. 6 illustrates an example of a process 600 for executing alert deduplication in response to receiving an alert notification from another device, in accordance with certain embodiments. In some embodiments, the process 600 can be executed, for example, by the therapy management engine 112 of FIGS. 1A-B and 2. In addition, or alternatively, the process 600 can be executed, for example, by the application 106 of FIGS. 1A-B and 2. In addition, or alternatively, the process 600 can be executed generally by any of the display devices 107 of FIGS. 1A-B and 2.

Although any number of systems, in whole or in part, can implement the process 600, to simplify discussion, the process 600 will be described as being performed by the application 106 of FIGS. 1A-B and 2, with particular reference to the system 300 of FIG. 3. With reference to the system 300 of FIG. 3, in some aspects, the process 600 can be executed continuously and in parallel by each of the applications 106-1 and 106-2. In addition, or alternatively, the process 600 can be executed by the application 106-1 and the application 106-2 upon the occurrence or determination of an event or condition, such as receipt of analyte data or an alert from the analyte sensor system 104 or another device or system. Other variations will be apparent to one skilled in the art after a detailed review of the present disclosure.

At block 602, the application 106 receives an alert notification from another device. The alert notification may be sent, for example, according to that other device's execution of the block 516 of FIG. 5A or the block 568 of FIG. 5B. In an example, if the application 106 corresponds to the application 106-1 of FIG. 3, the application 106-1 can receive the alert notification from the display device 107c. In another example, if the application 106 corresponds to the application 106-2 of FIG. 3, the application 106-2 can receive the alert notification from the display device 107a. The alert notification can include (and/or exclude) any of the data described relative to the block 516 of FIG. 5A.

At block 606, the application 106 determines an alert identifier for an alert associated with the alert notification. In some aspects, the alert identifier can be included with the alert notification, such that the block 606 includes retrieving the alert identifier from the alert notification. In some aspects, the alert notification can include information sufficient to generate the alert identifier. In some of these aspects, the block 606 can include generating the alert identifier using the information included with the alert notification, as discussed above relative to the block 506 of FIG. 5A.

At block 608, the application 106 automatically compares the alert identifier generated at the block 606 to a stored record of historical alerts previously processed at the display device 107. In general, the block 608 can include performing any of the functionality discussed relative to the block 406 of FIG. 4 and/or the block 508 of FIG. 5A.

At decision block 610, the application 106 determines whether the alert identifier for the alert matches an alert identifier for any of the historical alerts of the stored record. As discussed previously relative to FIG. 4, the existence of the alert identifier in the stored record can signify that the display device 107 has already presented the same alert. The display device 107 may have already presented the same alert, for example, as a result of having previously generated an alert at the display device 107, as discussed above relative to FIG. 5A. In certain aspects, blocks 610, 612, and 618 together serve as an example of conditional suppression as discussed relative to the block 408 of FIG. 4.

If it is determined, at the decision block 610, that the alert identifier for the alert matches an alert identifier for a historical alert of the stored record, at block 618, the application 106 suppresses the alert, thereby avoiding presentation of a redundant alert. The block 618 can include, for example, executing any of the functionality discussed above relative to the block 518 of FIG. 5A. If it is determined, at the decision block 610, that the alert identifier does not match an alert identifier for any historical alert of the stored record, at block 612, the application 106 causes or allows the alert to be presented, for example, as discussed above relative to the block 512 of FIG. 5A. At block 614, the application 106 updates the stored record of historical alerts, for example, as discussed above relative to the block 514 of FIG. 5A. After either block 614 or block 618, the process 600 ends.

FIG. 7 illustrates an example of a process 700 for synchronizing alert state between devices, in accordance with certain embodiments. In some embodiments, the process 700 can be executed, for example, by the therapy management engine 112 of FIGS. 1A-B and 2. In addition, or alternatively, the process 700 can be executed, for example, by the application 106 of FIGS. 1A-B and 2. In addition, or alternatively, the process 700 can be executed generally by any of the display devices 107 of FIGS. 1A-B and 2.

Although any number of systems, in whole or in part, can implement the process 700, for clarity, the process 700 will be described relative to two devices that are referred to as Device #1 and Device #2. In some aspects, Device #1 and Device #2 may correspond to the display devices 107a and 107c, respectively, of FIG. 3. In some aspects, Device #1 and Device #2 may correspond to the display devices 107c and 107a, respectively, of FIG. 3. Other examples will be apparent to one skilled in the art after a detailed review of the present disclosure.

In addition, although the process 700 is described relative to devices, it should be appreciated that the process 700 may be facilitated by respective software applications on such devices that operate as described relative to the application 106 of FIGS. 1A-B and 2. For example, in some aspects, with reference to FIG. 3, the process 700 can be executed continuously and in parallel by each of the applications 106-1 and 106-2. In addition, or alternatively, the process 700 can be executed by the application 106-1 and the application 106-2 upon the occurrence or determination of an event or condition, such as receipt of analyte data or an alert from the analyte sensor system 104 or another device or system. Other variations will be apparent to one skilled in the art after a detailed review of the present disclosure.

At block 702, Device #1 causes or allows an alert to be presented thereon, for example, as described relative to the block 512 of FIG. 5A or the block 612 of FIG. 6. At block 704, Device #1 determines a user response to the alert. The user response can be determined in relation to a timestamp, for example, according to a system time that is synchronized between Device #1 and Device #2.

In some aspects, the user response can be, or can include, user acknowledgement of the alert via an interface provided thereon. The user acknowledgement can be received, for example, via button press, swipe on a touchscreen, interaction with a graphical user interface, etc. In addition, or alternatively, the user response can be, or can include, user feedback indicating treatment in response to the alert. For example, in a diabetes treatment context, the user feedback can indicate insulin administered, activity undertaken, glucose ingested, or the like. In some aspects, the determined response can be, or can indicate, the absence of any acknowledgement or other action by the user over a predetermined time period following the alert (e.g., 10 minutes).

In addition, or alternatively, the user response can be, or can include, discovered information indicating treatment in response to the alert. In an example involving a diabetes treatment context, Device #1 can discover that was insulin administered following presentation of a high glucose alert. The insulin administration can be discovered, for example, via a data source providing insulin data (e.g., via a connection to a smart pen, insulin pump, etc.). In some aspects, Device #1 may be, for example, an insulin pump, thus providing immediate and direct access to such data. In another example involving a diabetes treatment context, Device #1 can discover that that activity was undertaken following presentation of a high glucose alert. The activity can be discovered, for example, via activity data available on Device #1 (e.g., activity data indicating a walk or run). In another example involving a diabetes treatment context, Device #1 can discover glucose ingestion following presentation of a low glucose alert. The glucose ingestion can be discovered, for example, based on meal data available to Device #1, a detected glucose increase, a detected change in glucose trend, etc.

At block 706, Device #1 updates an alert state for the alert based on the user response to the alert. The update can include or indicate, for example, a user response to a given alert, such as whether the alert has been acknowledged by the user (e.g., via a button press or swipe), whether the alert has been responded to with treatment, and/or the like. The updated alert state can include, for example, an alert identifier, as discussed above, and a timestamp associated with the user response (e.g., a time of acknowledgement). In addition, or alternatively, the updated alert state can include an urgency status based on the timestamp associated with the user response. In certain aspects, the urgency status can indicate multiple levels of escalation based on the timestamp to support, for example, escalating alarms (e.g., adjustments to volume, brightness, frequency, etc.).

The update to the alert state can be made, for example, in the stored record of historical alerts on or accessible to Device #1. For example, if the response is acknowledgement or treatment, Device #1 can remove or clear the alert, update a field for the alert status, or the like. In some aspects, treatment can be considered the same as acknowledgement, with the same update being performed in either scenario. In other aspects, treatment (and/or treatment type) can be a separately tracked state that may be considered differently (e.g., an alert may be cleared for treatment but not for acknowledgement).

Following block 706, Device #1 may execute blocks 708 and 710. At block 708, Device #1 notifies a remote target of the updated alert state. The remote target may be, for example, the server system 330 of FIG. 3. In certain aspects, notification of the remote target can facilitate, for example, publication of the updated alert state to a supporter device such as the supporter device 311 of FIG. 3. At block 710, Device #1 notifies another device (e.g., Device #2) of the updated alert state. The notification can include, for example, the alert identifier for the alert, an indication of the updated alert state (e.g., an explicit indication of the user's response to the alert, such as acknowledgement or treatment), the associated timestamp discussed above, and/or other information related to the alert or the user response to the alert. In some aspects, as a performance optimization, the notification can include the alert identifier and an indication of the updated alert state, to the exclusion of other data about the alert or response, thereby preserving network bandwidth.

At block 712, Device #2 receives the notification of the updated alert state. At block 714, Device #2 determines the alert identifier for the alert associated with the updated alert state, for example, by retrieving the alert identifier from the notification. At block 716, Device #2 updates the alert state for the alert based on the user response to the alert. The update can be made, for example, in the stored record of historical alerts on or accessible to Device #2, in similar fashion to the update discussed relative to the block 706. After block 716, the process 700 ends.

FIG. 8 illustrates an example of a process 800 for adjusting alert timing in response to an updated alert state, in accordance with certain embodiments. In some embodiments, the process 800 can be executed, for example, by the therapy management engine 112 of FIGS. 1A-B and 2. In addition, or alternatively, the process 800 can be executed, for example, by the application 106 of FIGS. 1A-B and 2. In addition, or alternatively, the process 800 can be executed generally by any of the display devices 107 of FIGS. 1A-B and 2. Although any number of systems, in whole or in part, can implement the process 800, for clarity, the process 800 will be described relative to Device #2, as referenced above relative to FIG. 7. In certain aspects, the process 800 may be executed in parallel to the process 700 of FIG. 7.

At block 802, Device #2 maintains one or more timers for presenting a current alert. In an example, Device #2 can maintain a timer to delay a first presentation of the current alert for a period of time. For example, the current alert may only presented if no updated state (e.g., acknowledgement or treatment) is received from another device, such as Device #1, within the period of time. In another example, Device #2 can maintain a timer for re-presenting the current alert if no acknowledgement of the current alert has been received on either Device #1 or Device #2. In another example, Device #2 can maintain a timer corresponding to a threshold period of time, for example, such that the current alert is presented only if the user's analyte values exceed a threshold value for the threshold period of time. Other examples of timers that may be maintained by Device #2 will be apparent to one skilled in the art after a detailed review of the present disclosure.

At block 804, Device #2 receives a notification of an updated alert state for the current alert. The notification of the updated alert state may be received, for example, from Device #1, as discussed relative to the block 712 of FIG. 7. At block 806, Device #2 determines an alert identifier for an alert associated with the updated alert state, for example, by retrieving the alert identifier from the notification. The block 806 can include, for example, matching the alert identifier from the notification to the alert identifier for the current alert.

At block 808, Device #2 resets the one or more timers for the current alert to correspond to a timestamp associated with the updated alert state (e.g., a time of acknowledgement). For example, if Device #2 would normally present, or re-present, the current alert after five minutes, the five-minute timer can be reset to start in correspondence to the timestamp associated with the updated alert state (e.g., at the time of acknowledgement). In other aspects, the timer(s) and/or the current alert can be cleared, for example, in response to the updated alert state, such that no presentation, or re-presentation, of the current alert occurs. After block 808, the process 800 ends.

FIG. 9 illustrates an example of a process 900 for adapting to device modes that impact alert presentation, in accordance with certain embodiments. In some embodiments, the process 900 can be executed, for example, by the therapy management engine 112 of FIGS. 1A-B and 2. In addition, or alternatively, the process 900 can be executed, for example, by the application 106 of FIGS. 1A-B and 2. In addition, or alternatively, the process 900 can be executed generally by any of the display devices 107 of FIGS. 1A-B and 2.

Although any number of systems, in whole or in part, can implement the process 900, for clarity, the process 900 will be described relative to two devices that are referred to as Device #1 and Device #2. In some aspects, Device #1 and Device #2 may correspond to the display devices 107a and 107c, respectively, of FIG. 3. In some aspects, Device #1 and Device #2 may correspond to the display devices 107c and 107a, respectively, of FIG. 3. Other examples will be apparent to one skilled in the art after a detailed review of the present disclosure. In addition, although the process 900 is described relative to devices, it should be appreciated that the process 900 may be facilitated by respective software applications on such devices that operate as described relative to the application 106 of FIGS. 1A-B and 2.

At block 902, Device #2 operates in a DND mode, or in a similar mode that prevents alert presentation. Device #2 may operate in the DND mode, for example, in response to a user command. At block 904, Device #1 determines to present an alert associated with elevated urgency, for example, according to predetermined urgency criteria. The predetermined urgency criteria can relate, for example, to measured analyte levels over any past period of time and/or predicted levels over any future period of time. The predetermined urgency criteria can be specified, for example, in terms of a high threshold (e.g., a threshold associated with hyperglycemia), a low threshold (e.g., a threshold associated with hypoglycemia), a failure to fall within a preferred range of analyte levels (e.g., a range associated with euglycemia or a user-defined range), a time period over which any of the foregoing are satisfied (e.g., a threshold period of time), a rate of change, combinations of the foregoing and/or the like.

At block 906, Device #1 identifies that previous alert presentations (e.g., alerts presented within the last 30 minutes) have not been acknowledged by a user, for example, at Device #1 or Device #2. The previous alert presentations may be for the same alert and/or different alerts. In general, the fact that the previous alert presentations have not been acknowledged may be indicative of a need to overcome a DND mode on Device #2.

At block 908, Device #1 notifies Device #2 of the alert and the elevated urgency so as to overcome the DND mode and cause presentation of the alert at Device #2. In some aspects, the notification can further include an explicit indication of the lack of acknowledgement at Device #1. At block 910, Device #2 receives the notification of the alert and the elevated urgency. At block 912, Device #2 escalates an alarm to overcome the DND mode and present the alert at Device #2. After block 912, the process 900 ends.

FIG. 10A illustrates an example of a process 1000 for delaying transmission of an updated alert state, in accordance with certain embodiments. In certain aspects, the process 1000 modifies operation of the process 700 of FIG. 7 to accommodate a scenario which communication between devices is interrupted. In some embodiments, the process 1000 can be executed, for example, by the therapy management engine 112 of FIGS. 1A-B and 2. In addition, or alternatively, the process 1000 can be executed, for example, by the application 106 of FIGS. 1A-B and 2. In addition, or alternatively, the process 1000 can be executed generally by any of the display devices 107 of FIGS. 1A-B and 2.

Although any number of systems, in whole or in part, can implement the process 1000, for clarity, the process 1000 will be described relative to two devices that are referred to as Device #1 and Device #2. In some aspects, Device #1 and Device #2 may correspond to the display devices 107a and 107c, respectively, of FIG. 3. In some aspects, Device #1 and Device #2 may correspond to the display devices 107c and 107a, respectively, of FIG. 3. Other examples will be apparent to one skilled in the art after a detailed review of the present disclosure.

In addition, although the process 1000 is described relative to devices, it should be appreciated that the process 1000 may be facilitated by respective software applications on such devices that operate as described relative to the application 106 of FIGS. 1A-B and 2. For example, in some aspects, with reference to FIG. 3, the process 1000 can be executed continuously and in parallel by each of the applications 106-1 and 106-2. In addition, or alternatively, the process 1000 can be executed by the application 106-1 and the application 106-2 upon the occurrence or determination of an event or condition, such as receipt of analyte data or an alert from the analyte sensor system 104 or another device or system. Other variations will be apparent to one skilled in the art after a detailed review of the present disclosure.

At block 1002, Device #1 causes or allows an alert to be presented thereon, for example, as described relative to the block 702 of FIG. 7. At block 1004, Device #1 determines a user response to the alert, for example, as described relative to the block 704 of FIG. 7. At block 1006, Device #1 updates an alert state for the alert based on the user response to the alert, for example, as discussed relative to the block 706 of FIG. 7.

At decision block 1007, Device #1 determines whether Device #2 is currently connected thereto. In some aspects, Device #1 can determine whether Device #2 is currently connected thereto, for example, by periodically pinging Device #2. If it is determined, at the decision block 1007, that Device #1 and Device #2 are not currently connected to each other, such that communication between the two devices is interrupted, the process 1000 proceeds to block 1008. At block 1008, Device #1 caches the updated alert state for later transmission, such that the updated alert state can be transmitted to Device #2, for example, upon reestablishment of a connection therewith. The block 1008 can include, for example, initiating a process to transmit cached alert states to Device #2 upon reconnection with Device #2, as will be discussed in greater detail relative to FIG. 10B.

If it is determined, at the decision block 1007, that Device #1 and Device #2 are currently connected to each other, at block 1010, Device #1 notifies Device #2 of the updated alert state, as discussed relative to the block 710 of FIG. 7. At block 1012, Device #2 receives the notification of the updated alert state. At block 1014, Device #2 determines the alert identifier for the alert associated with the updated alert state, for example, by retrieving the alert identifier from the notification. At block 1016, Device #2 updates the alert state for the alert based on the user response to the alert, as discussed relative to the block 716 of FIG. 7. After either block 1008 or 1016, the process 1000 ends.

FIG. 10B illustrates an example of a process 1050 for transmitting cached alert states to a device upon reconnection with the device, in accordance with certain embodiments. In certain aspects, the process 1050 can be triggered, for example, at the block 1008 of FIG. 10A. In some embodiments, the process 1050 can be executed, for example, by the therapy management engine 112 of FIGS. 1A-B and 2. In addition, or alternatively, the process 1050 can be executed, for example, by the application 106 of FIGS. 1A-B and 2. In addition, or alternatively, the process 1050 can be executed generally by any of the display devices 107 of FIGS. 1A-B and 2.

Although any number of systems, in whole or in part, can implement the process 1050, for clarity, the process 1050 will be described relative to two devices that are referred to as Device #1 and Device #2. In some aspects, Device #1 and Device #2 may correspond to the display devices 107a and 107c, respectively, of FIG. 3. In some aspects, Device #1 and Device #2 may correspond to the display devices 107c and 107a, respectively, of FIG. 3. Other examples will be apparent to one skilled in the art after a detailed review of the present disclosure.

In addition, although the process 1050 is described relative to devices, it should be appreciated that the process 1050 may be facilitated by respective software applications on such devices that operate as described relative to the application 106 of FIGS. 1A-B and 2. For example, in some aspects, with reference to FIG. 3, the process 1050 can be executed continuously and in parallel by each of the applications 106-1 and 106-2. In addition, or alternatively, the process 1050 can be executed by the application 106-1 and the application 106-2 upon the occurrence or determination of an event or condition, such as receipt of analyte data or an alert from the analyte sensor system 104 or another device or system. Other variations will be apparent to one skilled in the art after a detailed review of the present disclosure.

At decision block 1052, Device #1 determines whether Device #2 is currently connected thereto, as discussed relative to the decision block 1007 of FIG. 10A. If it is determined, at the decision block 1052, that Device #1 and Device #2 are not currently connected to each other, such that communication between the two devices remains interrupted, the process 1050 remains at the decision block 1052.

If it is determined, at the decision block 1052, that Device #1 and Device #2 are currently connected to each other, at decision block 1054, Device #1 determines whether a cache of updated alert states is empty. If it is determined, at the decision block 1054, that the cache of updated alert states is empty, the process 1050 ends. However, if it is determined, at the decision block 1054, that the cache of updated alert states is non-empty, at block 1056, Device #1 sends, to Device #2, a notification that includes the contents of the cache. From block 1056, the process 1050 can proceed to block 1058 and block 1068 in parallel.

At block 1058, Device #2 receives the notification including the cached updated alert states. At block 1060, Device #2 determines the alert identifier for the alert associated with each updated alert state, for example, by retrieving each alert identifier from the notification. At block 1062, Device #2 updates the alert state of each corresponding alert, as discussed relative to the block 716 of FIG. 7. At block 1068, Device #1 clears the cache. After either block 1062 or 1068, the process 1050 ends.

FIG. 11 illustrates an example of a process 1100 for synchronizing settings between devices, in accordance with certain embodiments. In some embodiments, the process 1100 can be executed, for example, by the therapy management engine 112 of FIGS. 1A-B and 2. In addition, or alternatively, the process 1100 can be executed, for example, by the application 106 of FIGS. 1A-B and 2. In addition, or alternatively, the process 1100 can be executed generally by any of the display devices 107 of FIGS. 1A-B and 2.

Although any number of systems, in whole or in part, can implement the process 1100, for clarity, the process 1100 will be described relative to two devices that are referred to as Device #1 and Device #2. In some aspects, Device #1 and Device #2 may correspond to the display devices 107a and 107c, respectively, of FIG. 3. In some aspects, Device #1 and Device #2 may correspond to the display devices 107c and 107a, respectively, of FIG. 3. Other examples will be apparent to one skilled in the art after a detailed review of the present disclosure. In addition, although the process 1100 is described relative to devices, it should be appreciated that the process 1100 may be facilitated by respective software applications on such devices that operate as described relative to the application 106 of FIGS. 1A-B and 2.

At block 1102, Device #1 receives a change to settings thereon. In an example, the change can be to alert settings. In various aspects, the alert settings may provide for alerting a user, for example, if the user's analyte values exceed a threshold value for a threshold period of time. According to these aspects, the change may relate, for example, to the threshold value and/or the threshold period of time. In various aspects, the alert settings may also include parameters providing for whether and how an alert is presented, such as sound, volume, vibration, and silence, where each may vary for different times and/or analyte values. According to these aspects, the change may relate, for example, such parameters providing for whether and how an alert is presented. In certain aspects, the change to alert settings can be received, for example, from a user of Device #1 via a graphical user interface provided by the device. In other examples, the change relate to can relate to user interface appearance (e.g., colors) and/or application functionality. Other examples of changes to settings will be apparent to one skilled in the art after a detailed review of the present disclosure.

At block 1104, Device #1 saves an updated settings state, for example, in memory thereon, in association with a timestamp of the change. The updated settings state can indicate, for example, the change(s), a complete set of settings, and/or the like. As discussed previously, in some aspects, the updated settings state can be, or can include, an alert settings state, such that the alert settings state can indicate the change(s) and a complete set of alert settings.

Following block 1104, Device #1 may execute blocks 1106 and 1108. At block 1106, Device #1 notifies a remote target of the updated settings state. The remote target, may be, for example, the server system 330 of FIG. 3. In certain aspects, notification of the remote target can facilitate, for example, sharing of the updated settings state to a supporter device such as the supporter device 311 of FIG. 3. At block 1108, Device #1 notifies another device (e.g., Device #2) of the updated settings state. The notification can include, for example, the updated settings state and the associated timestamp.

At block 1110, Device #2 receives the notification of the updated settings state. At block 1112, Device #2 determines a most recent settings state. For example, Device #2 can compare the timestamp associated with the updated settings state with a timestamp associated with a current settings state of Device #2 to determine which state is more recent. At decision block 1114, Device #2 determines whether the updated settings state from Device #1 is the most recent settings state available to Device #2 (e.g., more recent than the current settings state). If Device #2 determines, at the decision block 1114, that the updated settings state from Device #1 is not the most recent settings state, the process 1100 ends without the updated settings state being implemented at Device #2. However, if Device #2 determines, at the decision block 1114, that the updated settings state from Device #1 represents the most recent settings state, Device #2 implements the updated settings state at block 1116. After block 1116 the process 1100 ends.

In certain aspects, sharing and synchronizing settings, as discussed above relative to the process 1100 of FIG. 11, can enable settings roll-back functionality. Device #1 and/or Device #2 can roll back to settings states associated with earlier timestamps, for example, in response to direction from a user, in response to an automatic determination that another settings state resulted in better patient health (e.g., based on time in range or other metrics), and/or the like.

FIG. 12 illustrates an example of a process 1200 for generating and implementing alert analytics, in accordance with certain embodiments. In some embodiments, the process 1200 can be executed, for example, by the therapy management engine 112 of FIGS. 1A-B and 2. In addition, or alternatively, the process 1200 can be executed, for example, by the application 106 of FIGS. 1A-B and 2. In addition, or alternatively, the process 1200 can be executed generally by any of the display devices 107 of FIGS. 1A-B and 2. Although any number of systems, in whole or in part, can implement the process 1200, to simplify discussion, the process 1200 will be described as being performed by the application 106 of FIGS. 1A-B and 2, with particular reference to the system 300 of FIG. 3.

At block 1202, the application 106 generates analytics based on settings and alert activity. For example, the analytics can be based on the stored record historical alerts discussed relative to FIG. 3. In some aspects, the analytics can include metrics describing a user's level of engagement with alerts presented at the display device 107 and/or other receiver devices (e.g., number or percentage of alerts acknowledged, on which devices alerts were acknowledged or acknowledged, etc.). The metrics can further consider, for example, a time and/or location associated with the user's engagement (e.g., time and/or location when alerts are acknowledged versus not acknowledged, time and/or location in association with a device at which acknowledgement occurs, etc.). In addition, or alternatively, the analytics can include recommended changes to settings, for example, based on the analytics related to the level of engagement (e.g., based on rules, machine-learning models, or the like).

In certain aspects, the analytics can facilitate personalized optimization of alert settings to decrease alert fatigue and increase user engagement. In certain aspects, low user engagement for a given user (e.g., a percentage of acknowledged alerts is below a threshold) could indicate that there are too many alerts to be useful to the user. According to this example, the application 106 can determine an alert settings optimization that raises thresholds, for example, to decrease the likely number of alerts that are presented.

In another example, the alert analytics can relate to establishing a priority device for alert presentation based on user behavior (e.g., based on time and/or location). The priority device may present alerts first among a set of user devices, for example, to save processing at the other devices and increase user engagement. If the application 106 determines, for example, based on the level of user engagement discussed above, that alerts are consistently acknowledged at one device over another, the analytics generated at the block 1202 can include a determination that the device at which alerts are consistently acknowledged should be made the priority device. The priority device can also be made variable, for example, based on time, location, and/or other dynamic factors. For example, as part of the analytics generated at the block 1202, the application 106 can determine a set of conditions (e.g., time or location) under which each of a set of devices should be the priority device, such that a change in conditions (e.g., change in time or location) can trigger a change of the priority device. For example, the application 106 can determine that one device should be made the priority device for presentation of alerts if it is determined that such device is generally with the user at a given time, such as every day at noon when the user is at the pool.

At block 1204, the application 106 implements one or more changes to settings at the display device 107 based on the analytics generated at the block 1202. In some aspects, the changes to settings may be presented to a user for approval in advance of implementation. In some aspects, the change to settings may be shared with other devices, for example, as discussed relative to the process 1100 of FIG. 11. After block 1204, the process 1200 ends.

FIG. 13 illustrates an example of a process 1300 for adapting alert settings to a current user, in accordance with certain embodiments. In some embodiments, the process 1300 can be executed, for example, by the therapy management engine 112 of FIGS. 1A-B and 2. In addition, or alternatively, the process 1300 can be executed, for example, by the application 106 of FIGS. 1A-B and 2. In addition, or alternatively, the process 1300 can be executed generally by any of the display devices 107 of FIGS. 1A-B and 2.

Although any number of systems, in whole or in part, can implement the process 1300, to simplify discussion, the process 1300 will be described as being performed by the application 106 of FIGS. 1A-B and 2, with particular reference to the system 300 of FIG. 3. With reference to the system 300 of FIG. 3, in some aspects, the process 1300 can be executed continuously and in parallel by each of the applications 106-1 and 106-2. In addition, or alternatively, the process 450 can be executed by the application 106-1 and the application 106-2 at any suitable interval (e.g. every 5 minutes, every 10 minutes, every 15 minutes, etc.). Other variations will be apparent to one skilled in the art after a detailed review of the present disclosure.

At decision block 1302, the application 106 determines whether a current user of the display device 107 is a target user for alerts (e.g., the user 102). In various aspects, multiple users may be operating devices connected to the analyte sensor system 104. In an example, a device may have a different user than is typical (e.g., a child using a parent's device). In another example, the different devices may correspond to different users (e.g., two family members each have a device connected to the analyte sensor). In this context, it may be desirable to override certain features that may result in delay or suppression of redundant alert presentation, for example, according to FIGS. 4-6. The overridden features can include, for example, any action that is based on an updated alert state at a device not currently being used by a target user. Overriding such features can ensure that alert presentations are not missed by the target user.

In various aspects, the determination, at the decision block 1302, of whether the current user of the display device 107 can be made, for example, based on login information, various biometrics (e.g., facial recognition), or other information. In addition, or alternatively, the determination can be made based on a mode of operation, such as a “child mode” or “public mode” initiated by the target user. If it is determined, at decision block 1302, that the current user of the display device 107 is the target user for alerts (e.g., the user 102), the process 1300 ends without modifying alerting functionality. However, if it is determined, at the decision block 1302, that the current user of the display device 107 is not the target user for alerts, the process 1300 proceeds to block 1304.

At block 1304, the application 106 modifies (e.g., temporarily modifies) alerting functionality at the display device 107. In some aspects, the modifying can include suppressing all alerts at the display device 107, for example, so that the non-target user does not view and/or acknowledge such alerts. In addition, or alternatively, the modifying can include overriding alert suppression features.

In an example of overriding alert suppression features, in some aspects, the application 106 can override the removal or clearing of alerts based on acknowledgements and/or other alert state updates at the display device 107. According to this example, acknowledgements and/or other alert state updates at the display device 107 can instead be ignored, not recorded, and/or not substantively acted upon. In this way, when the display device 107 is returned to the target user, previous alerts can still be viewable and/or re-presentation of alerts can still occur.

In another example of overriding alert suppression features, in some aspects, the application 106 can implement a change that alert state updates are not shared with other devices. According to this example, alert suppression and/or delays on the other devices based on such updates may be averted, thereby decreasing a likelihood that the target user misses presentation of an alert.

In another example of overriding alert suppression features, in some aspects, any existing suppression of sharing of alerts with other devices can be overridden (e.g., turned off) so as to maximize exposure to the target user. In some aspects, the application 106 can cause the display device 107 to immediately share alerts with another device associated with the target user, even if such sharing would not ordinarily occur.

After block 1304, the process 1300 ends. In certain aspects, the application 106 can continue operating with the modified alerting functionality until it is determined that the current user of the display device 107 is the target user (e.g., during a future iteration of the process 1300). As discussed previously, the determination can be made based on login information, various biometrics (e.g., facial recognition), or other information. In addition, or alternatively, the determination can be made based on exiting a mode of operation that brought about the modified alerting functionality (e.g., by exiting “child mode” or “public mode.”).

FIG. 14 is a block diagram depicting a computer system 1400 configured for alert optimization in multi-receiver environments, for example, according to certain embodiments disclosed herein. Although depicted as a single physical device, in embodiments, the computer system 1400 may be implemented using virtual device(s), and/or across a number of devices, such as in a cloud environment and/or via separate modules of portable or cloud devices. As illustrated, the computer system 1400 includes a processor 1405, a memory 1410, a storage 1415, a network interface 1425, and one or more I/O interfaces 1420. In the illustrated embodiment, the processor 1405 retrieves and executes programming instructions stored in the memory 1410, as well as stores and retrieves application data residing in the storage 1415. The processor 1405 is generally representative of a single CPU and/or GPU, multiple CPUs and/or GPUs, a single CPU and/or GPU having multiple processing cores, and the like.

The memory 1410 is generally included to be representative of a random access memory (RAM). The storage 1415 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, the I/O devices 1435 (such as keyboards, monitors, etc.) can be connected via the I/O interface(s) 1420. Further, via the network interface 1425, the computer system 1400 can be communicatively coupled with one or more other devices and components, such as the user database 110. In certain embodiments, the computer system 1400 is communicatively coupled with other devices via a network, which may include the Internet, local network(s), and the like. The network may include wired connections, wireless connections, or a combination of wired and wireless connections. As illustrated, the processor 1405, memory 1410, storage 1415, network interface(s) 1425, and the I/O interface(s) 1420 are communicatively coupled by one or more interconnects 1430. In certain embodiments, the computer system 1400 is representative of the display device 107 associated with the user. In certain embodiments, as discussed above, the display device 107 can include the user's laptop, computer, smartphone, and the like. In another embodiment, the computer system 1400 is a server executing in a cloud environment.

In the illustrated embodiment, the storage 1415 includes the user profile 118, a stored record 1440, and settings 1442. The stored record 1440 can correspond, for example, to the stored record of historical alerts, as discussed relative to FIGS. 3-12. The settings 1442 can correspond, for example, to the settings (e.g., alert settings) discussed relative to FIGS. 3-12.

The memory 1410 includes the therapy management engine 112. The therapy management engine 112 can be executed by the computer system 1400 to perform operations, for example, of the process 400 of FIG. 4, the process 500 of FIG. 5A, the process 550 of FIG. 5B, the process 600 of FIG. 6, the process 700 of FIG. 7, the process 800 of FIG. 8, the process 900 of FIG. 9, the process 1000 of FIG. 10A, the process 1050 of FIG. 10B, the process 1100 of FIG. 11, the process 1200 of FIG. 12, and/or the process 1300 of FIG. 13. Each of these non-limiting examples can stand on its own or can be combined in various permutations or combinations with one or more of the other examples.

In certain aspects, a method of alert optimization includes receiving, at a first device, one or more analyte measurements produced by an analyte sensor worn by a patient. The method also includes determining, at the first device, a first unique identifier for a first alert based on the one or more analyte measurements. The method also includes comparing, at the first device, the first unique identifier for the first alert to a stored record that includes unique identifiers for historical alerts. The method also includes conditionally suppressing the first alert on the first device based on the comparing.

In certain aspects, a method of alert optimization includes receiving, at a first device, an alert notification from a second device, where the alert notification includes a unique identifier for an alert, and where the alert is based on one or more analyte measurements produced by an analyte sensor worn by a patient. The method also includes comparing, at the first device, the unique identifier to a stored record that includes unique identifiers for historical alerts. The method also includes conditionally suppressing the alert on the first device based on the comparing. Other embodiments of this aspect include corresponding computer systems, apparatus, and computer programs recorded on one or more computer storage devices, each configured to perform the actions of the methods.

In certain aspects, a method of synchronizing alert state between devices includes presenting, at a first device, an alert related to one or more analyte measurements. The method also includes determining, at the first device, a user response to the alert. The method also includes updating, by the first device, a state of the alert in a stored record of alerts based on the user response, where the user response may include at least one of a user acknowledgement of the alert or an indication of treatment in response to the alert. The method also includes notifying at least one of a second device or a remote target of the updated state of the alert. Other embodiments of this aspect include corresponding computer systems, apparatus, and computer programs recorded on one or more computer storage devices, each configured to perform the actions of the methods.

In certain aspects, a method of adjusting alert timing according to alert state includes receiving, at a first device, an updated state of an alert from a second device, where the alert relates to one or more analyte measurements of a patient. The method also includes updating, at the first device, a stored record of alerts based on the updated state of the alert. The method also includes, responsive to the updated state of the alert, resetting a timer for presenting the alert at the first device. Other embodiments of this aspect include corresponding computer systems, apparatus, and computer programs recorded on one or more computer storage devices, each configured to perform the actions of the methods.

In certain aspects, a method of adapting notifications to a device mode of another device includes presenting, at a first device, an alert related to one or more analyte measurements. The method also includes determining, at the first device, a user response to the alert. The method also includes updating, by the first device, a state of the alert in a stored record of alerts based on the user response, where the user response includes at least one of a user acknowledgement of the alert or an indication of treatment in response to the alert. The method also includes notifying a second device of an elevated urgency associated with the alert to overcome a do-not-disturb mode at the second device. Other embodiments of this aspect include corresponding computer systems, apparatus, and computer programs recorded on one or more computer storage devices, each configured to perform the actions of the methods.

In certain aspects, a method of delayed transmission of alert state to another device includes presenting, at a first device, an alert related to one or more analyte measurements. The method also includes determining, at the first device, a user response to the alert. The method also includes updating, by the first device, a state of the alert in a stored record of alerts based on the user response, where the user response includes at least one of a user acknowledgement of the alert or an indication of treatment in response to the alert. The method also includes caching the updated state of the alert for later transmission to a second device responsive to a determination that the first device is not currently connected to the second device. The method also includes notifying the second device of the updated state of the alert responsive to a determination that a connection with the second device has been reestablished. Other embodiments of this aspect include corresponding computer systems, apparatus, and computer programs recorded on one or more computer storage devices, each configured to perform the actions of the methods.

In certain aspects, a method of synchronizing settings includes receiving, at a first device, a change to settings on the first device. The method also includes saving an updated settings state indicating the change in association with a timestamp of the change. The method also includes notifying at least one of a second device or a remote target of the updated settings state. Other embodiments of this aspect include corresponding computer systems, apparatus, and computer programs recorded on one or more computer storage devices, each configured to perform the actions of the methods.

In certain aspects, a method of synchronizing settings includes receiving, at a first device, a notification from a second device of an updated settings state at the second device. The method also includes determining that the updated settings state is more recent than a current settings state at the first device. The method also includes, responsive to the determining, implementing the updated settings state at the first device. Other embodiments of this aspect include corresponding computer systems, apparatus, and computer programs recorded on one or more computer storage devices, each configured to perform the actions of the methods.

In certain aspects, a method of generating and implementing alert analytics includes generating, at a device, analytics based on settings on the device and alert activity on the device. The method also includes implementing a change to the settings on the device based on the analytics. Other embodiments of this aspect include corresponding computer systems, apparatus, and computer programs recorded on one or more computer storage devices, each configured to perform the actions of the methods.

In certain aspects, a method of adapting alert settings to a current user of a device includes determining whether the current user of the device is a target user for alerts related to analyte measurements. The method also includes, responsive to a determination that that the current user of the device is not a target user for alerts related to analyte measurements, overriding one or more alert suppression features at the device. Other embodiments of this aspect include corresponding computer systems, apparatus, and computer programs recorded on one or more computer storage devices, each configured to perform the actions of the methods.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method of alert optimization, the method comprising:
receiving, at a first device, one or more analyte measurements produced by an analyte sensor worn by a patient;
determining, at the first device, a first unique identifier for a first alert based on the one or more analyte measurements;
comparing, at the first device, the first unique identifier for the first alert to a stored record comprising unique identifiers for historical alerts; and
conditionally suppressing the first alert on the first device based on the comparing.

2. The method of claim 1, wherein the determining the first unique identifier comprises deterministically generating the first unique identifier based on a type of the first alert and a time associated with at least one of the one or more analyte measurements.

3. The method of claim 1, wherein the conditionally suppressing comprises suppressing the first alert responsive to a determination that the first unique identifier matches at least one of the unique identifiers for historical alerts.

4. The method of claim 1, further comprising determining that criteria associated with a type of the first alert is satisfied, wherein the determining of the first unique identifier is performed responsive to the determining that the criteria is satisfied.

5. The method of claim 1, wherein:
the conditionally suppressing comprises allowing presentation of the first alert at the first device responsive to a determination that the first unique identifier does not match any of the unique identifiers for historical alerts, and
the method further comprises updating the stored record to include the first unique identifier in the unique identifiers for historical alerts.

6. The method of claim 5, further comprising:
receiving, at the first device, an alert notification from a second device, the alert notification comprising a second unique identifier for a second alert;
comparing, at the first device, the second unique identifier to the updated stored record; and
suppressing the second alert responsive to a determination that the second unique identifier matches the first unique identifier.

7. The method of claim 5, further comprising:
receiving, at the first device, an alert notification from a second device, the alert notification comprising a second unique identifier for a second alert;
comparing, at the first device, the second unique identifier to the updated stored record; and
allowing presentation of the second alert at the first device responsive to a determination that the second unique identifier for the second alert does not match any of the unique identifiers for historical alerts.

8. The method of claim 5, further comprising:
determining a user response to the first alert; and
updating a state of the first alert in the stored record based on the determined user response.

9. The method of claim 8, wherein the user response comprises at least one of a user acknowledgement of the first alert or an indication of treatment in response to the first alert.

10. The method of claim 8, further comprising notifying at least one of a second device or a remote target of the updated state of the first alert.

11. The method of claim 10, wherein the notifying further comprises notifying the second device of an elevated urgency associated with the first alert to overcome a do-not-disturb mode at the second device.

12. The method of claim 8, further comprising caching the updated state of the first alert for later transmission to a second device responsive to a determination that the first device is not currently connected to the second device.

13. The method of claim 12, further comprising notifying the second device of the updated state of the first alert responsive to a determination that a connection with the second device has been reestablished.

14. The method of claim 1, further comprising:
receiving, from a second device, a notification of an updated alert state related to the first alert, the notification comprising the first unique identifier; and
updating, at the first device, the stored record based on the updated alert state.

15. The method of claim 14, wherein the updating comprising updating a state of the first alert in the stored record based on the updated alert state.

16. The method of claim 14, wherein the updating comprises clearing the first alert in the stored record.

17. The method of claim 14, further comprising, responsive to the updated alert state, resetting a timer for presenting the first alert at the first device.

18. The method of claim 17, wherein the resetting comprises resetting the timer to correspond to a timestamp associated with the updated alert state.

19. A system for alert optimization, comprising:
an analyte sensor configured to generate measurements associated with an analyte level of a patient;
a device in data communication with the analyte sensor and configured to:
receive one or more analyte measurements produced by the analyte sensor;
determine a unique identifier for a first alert based on the one or more analyte measurements;
compare the unique identifier for the first alert to a stored record comprising unique identifiers for historical alerts; and
conditionally suppress the first alert on the device based on the comparing.

20. A computer-program product comprising a non-transitory computer-usable medium having computer-readable program code embodied therein, the computer-readable program code adapted to be executed to implement a method of alert optimization, the method comprising:
receiving one or more analyte measurements produced by an analyte sensor worn by a patient;
determining a unique identifier for a first alert based on the one or more analyte measurements;
comparing the unique identifier for the first alert to a stored record comprising unique identifiers for historical alerts; and
conditionally suppressing the first alert on a device based on the comparing.
